(19) United States
(12) Patent Application Publication
(10) Pub. No.: US 2004/0043326 A1
(43) Pub. Date: Mar. 4, 2004

(54) Information recording medium and oxonol compound

(75) Inventors: Naoki Saito, Kanagawa (JP); Koji Wariishi, Kanagawa (JP); Michihiro Shibata, Kanagawa (JP)

Correspondence Address: SUGHRUE MION, PLLC, 2100 PENNSYLVANIA AVENUE, N.W., WASHINGTON, DC, 20037, US

(73) Assignee: FUJI PHOTO FILM CO. , LTD.

(21) Appl. No.: 10/455,723

(22) Filed: Jun. 6, 2003

Related U.S. Application Data
(63) Continuation of application No. 09/313,185, filed on May 18, 1999, now abandoned.

(30) Foreign Application Priority Data
May 19, 1998 (JP) 10-153877

(51) Int. Cl. G11B 7/24; C07D 409/00; C07D 411/00; C07D 333/64; C07D 333/72; C07D 409/02

(52) U.S. Cl. 430/270.14; 430/270.18; 546/281.1; 549/53; 549/54

(57) ABSTRACT

An optical information recording medium has a recording layer composed essentially of an oxonol compound of the following formula:

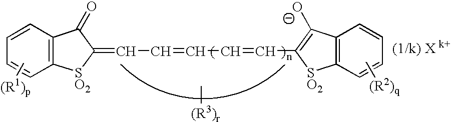

in which each of R¹, R² and R³ is a substituent group, X^{k+} is an onium ion, each of p and q is an integer of 0 to 4, r is an integer of 1 to 5, n is 0 or 1, k is an integer of 1 to 10, and in the case that p or q is an integer of 2 or more, plural substituent groups represented by R¹ or R² are the same or different or they can connect with each other to form a ring.

### FIELD OF THE INVENTION

This invention relates to an information recording medium on which information can be recorded by means of a laser beam. The invention also relates to a dye compound advantageously used for producing that medium.

### BACKGROUND OF THE INVENTION

An information recording medium (such as optical disc) on which information can be only once recorded by means of a laser beam is known as a recordable compact disc (CD-R), and is widely used, for example, as a data storage device for computers. The disc of CD-R has a multi-layered structure basically comprising a disc-shaped transparent substrate (usually made of polymer material such as polycarbonate) and a record layer provided thereon. Generally, a light-reflecting layer and a protective layer are further provided on the recording layer in order. The performance of CD-R greatly depends on characteristics of the recording layer, and hence various materials have been studied for the formation of the recording layer. Typical examples of those materials are (semi)metals (e.g., tellurium and indium) and organic dyes (e.g., polymethine and phthalocyanine).

The information can be recorded by irradiating the disc with a laser beam of near infrared region (usually 780 nm). By the application of the laser beam, the irradiated area of the recording layer is locally heated to change its physical or chemical characteristics, and pits are formed in the recording layer. Since the optical characteristics of the formed pits are different from those of the area having been not irradiated, the information is optically recorded. The recorded information can be read by a reproducing procedure comprising the steps of irradiating the recording layer with the same laser beam as that employed in the recording procedure, and detecting the difference of the optical characteristics (generally, light-reflection) between the pits and an area having no pits.

The recording layer comprising an organic dye (dye recording layer) can be formed by the steps of dissolving or dispersing the dye in an organic solvent to prepare a coating liquid, and applying the liquid on the substrate. Since those steps are much easier than the procedure for forming a metal recording layer, the dye recording layer is advantageous from the viewpoint of production cost. Further, the dye recording layer is more sensitive than the metal one. The dye recording layer, however, generally has poor durability to heat and light, and hence it has been desired to develop a dye recording layer having high resistance to both heat and light. Japanese Patent Provisional Publication No. 63(1988)-209995 discloses an information recording medium having a recording layer comprising an oxonol dye. That medium can keep reliable recording-reproducing characteristics for relatively long time, but the durability is still unsatisfactory. Consequently, it can be said that there is no organic dye recording layer having satisfactory durability.

Recently, data processing on computers has been performed at a high speed for processing an great amount of data. Accordingly, it has been more desired to develop an information recording medium suitable for the high speed recording and reproducing. For that medium, a recording layer having excellent recording characteristics is indispensable. Although the known oxonol dye layer for the optical information recording medium has relatively improved characteristics, it is desired to be further improved for the high speed data processing.

In addition, it is also desired to develop an information recording medium which can store data at a high recording density. For increasing the recording density, the laser beam is preferably focused on a spot as small as possible. Since the area of the spot can be reduced in proportion to the wavelength of the applied laser light, the laser emitting light of a short wavelength is theoretically advantageous. Accordingly, an optical disc irradiated with laser light of a wavelength shorter than 780 nm (which is a conventionally used wavelength) has been developed. A typical example is a recordable digital video disc (DVD-R), on which information can be recorded and reproduced with visible laser light (usually, having a wavelength of 600 to 700 nm). Thus, the DVD-R can store information at a relatively high density as compared with a conventional CD-R.

It is an object of the present invention to provide an information recording medium having both excellent recording characteristics and high durability (particularly against light). Further, it is another object of the invention to provide a novel oxonol dye compound advantageously employable for producing an optical information recording medium.

### SUMMARY OF THE INVENTION

The inventors studied on the substituent groups and the counter ions of known oxonol dye compounds, and finally invented an information recording medium improved in both the recording characteristics and durability.

The present invention resides in an information recording medium comprising a substrate and a recording layer provided thereon on which information can be recorded by irradiation with a laser beam, wherein the recording layer comprises an oxonol compound of the following general formula (1) in which at least one substituent group [(R³)ᵣ] is attached to at least one carbon chain of the methine chain of the formula.

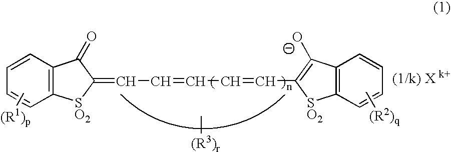

In the formula (1), each of R¹, R² and R³ independently represents a substituent group, X^{k+} represents an onium ion, each of p and q independently is an integer of 0 to 4, r is an integer of 1 to 5, n is 0 or 1, and k is an integer of 1 to 10. In the case where p or q is an integer of 2 or more, plural substituent groups represented by R¹ or R² may be the same or different or they may connect with each other to form a ring.

The preferred embodiments of the invention are as follows.

(1) In the formula (1), X^{k+} is a quaternary ammonium ion.

(2) In the formula (1), X^{k+} is an onium ion of the following formula (2):

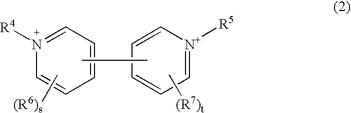

In the formula (2), each of R⁴ and R⁵ independently represents an alkyl group, an alkenyl group, an alkynyl group, an aryl group, or a heterocyclic group. Each of R⁶ and R⁷ independently represents a substituent group, and each of s and t independently represents an integer of 0 to 4. In the case where s or t is an integer of 2 or more, plural substituent groups represented by R⁶ or R⁷ may be the same or different or they may connect with each other to form a ring.

(3) A light-reflecting layer is further provided on the recording layer.

(4) The compound of the formula (1) is an oxonol compound having the following formula (3):

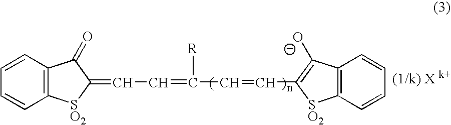

In the formula (3), R represents a substituent group such as methyl, ethyl, phenyl, 4-pyridyl, methoxy, or phenoxy. Further, n is 0 or 1, X^{k+} represents an onium ion, and k is 1 or 2.

### DETAILED DESCRIPTION OF THE INVENTION

The information recording medium of the invention is characterized by having a recording layer which comprises an oxonol compound of the following formula (1). The methine chain of the compound comprises at least one substituted carbon atom.

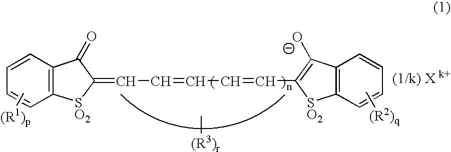

In the formula (1), each of R¹, R² and R³ independently represents a substituent group, X^{k+} represents an onium ion, each of p and q independently represents an integer of 0 to 4, r is an integer of 1 to 5, n is 0 or 1, and k is an integer of 1 to 10. In the case that p or q is an integer of 2 or more, plural substituent groups represented by R¹ or R² may be the same or different or they may connect with each other to form a ring.

With respect to the oxonol dye compound used in the invention, more detailed description is given below.

The oxonol compound of the invention consists essentially of an anion part (dye component) and a cation part (onium component). First, the anion part is described below.

In the formula (1), R¹, R² and R³ may be the same or different from each other. Each of them independently represents an alkyl group having 1-18 carbon atoms, an alkenyl group having 2-18 carbon atoms, an alkynyl group having 2-18 carbon atoms, an aryl group having 6-14 carbon atoms, a heterocyclic group having 4-9 carbon atoms, a halogen atom, nitro group, or cyano group. Further, each may be a group having the formula of —COR¹¹, —SO₂R¹¹, —SOR¹¹, —CO₂R¹¹, —OR¹¹, —SR¹¹, —OCOR¹¹, —OSO₂R¹¹, —CONR¹¹R¹², —SO₂NR¹¹R¹², —OCONR¹¹R¹², —OSO₂NR¹¹R¹², —NR¹¹R¹², —NR¹¹COR¹², —NR¹¹SO₂R¹², —NR¹¹—CO₂R¹², —NR¹¹CONR¹²R¹³, or —NR¹¹SO₂NR¹²R¹³. Each of R¹¹, R¹² and R¹³ independently represents an alkyl group having 1-18 carbon atoms, an alkenyl group having 2-18 carbon atoms, an alkynyl group having 2-18 carbon atoms, an aryl group having 6-14 carbon atoms, or a heterocyclic group having 4-9 carbon atoms.

The groups of R¹, R² and R³ may have one or more substituent groups. Examples of the substituent groups are the same as those described above for R¹, R² and R³.

Preferably, each of R¹ and R² is independently an alkyl group, an alkoxy group, or a halogen atom. As the alkyl group for R¹ or R², preferred is an alkyl group of straight or branched chain having 1-8 carbon atoms or a cyclic structure. More preferred is an alkyl group of (straight or branched) chain having 1-4 carbon atoms. Examples of the preferred alkyl groups include methyl, ethyl, n-propyl, isopropyl, n-butyl, sec-butyl, isobutyl, and tert-butyl. Particularly preferred is methyl group.

As the alkoxy group for R¹ or R², preferred is an alkoxy group of straight or branched chain having 1-8 carbon atoms or a cyclic structure. More preferred is an alkoxy group of straight or branched chain having 1-4 carbon atoms. Examples of the preferred alkoxy groups include methoxy, ethoxy, n-propoxy, isopropoxy, n-butoxy, sec-butoxy, isobutoxy, and tert-butoxy. Particularly preferred is methoxy group.

Examples of the preferred halogen atoms for R¹ or R² include fluorine atom, chlorine atom, and bromine atom. Particularly preferred are chlorine atom and bromine atom.

The substituent group represented by R³ in the formula (1) preferably is an alkyl group, an aryl group, a heterocyclic group, an alkoxy group, or an aryloxy group. As the alkyl group for R³, preferred is an alkyl group of straight or branched chain having 1-8 carbon atoms or a cyclic structure, and more preferred is an alkyl group of straight or branched chain having 1-4 carbon atoms. Examples of the preferred alkyl groups include methyl, ethyl, isopropyl, n-butyl, sec-butyl, isobutyl, and tert-butyl. Particularly preferred are methyl group and ethyl group.

As the aryl group for R³, preferred is an aryl group having 6-10 carbon atoms. Examples of the preferred aryl groups include phenyl, 1-naphthyl, and 2-naphthyl. Particularly preferred is phenyl group.

As the heterocyclic group for R³, preferred is a saturated or unsaturated heterocyclic group having 4-7 carbon atoms. Preferred hetero-atoms contained in the heterocyclic group are nitrogen atom, oxygen atom, and sulfur atom. Examples of the preferred heterocyclic groups include 4-pyridyl, 2-pyridyl, 2-pyrazyl, 2-imidazolyl, 2-furyl, 2-thiophenyl, 2-benzoxazolyl, and 2-benzothioxazolyl. Particularly preferred is 4-pyridyl.

As the alkoxy group for R³, preferred is an alkoxy group of straight or branched chain having 1-8 carbon atoms or a cyclic structure, and more preferred is an alkoxy group of straight or branched chain having 1-4 carbon atoms. Examples of the preferred alkoxy groups include methoxy, ethoxy, n-propoxy, isopropoxy, n-butoxy, sec-butoxy, isobutoxy, and tert-butoxy. Particularly preferred is methoxy group.

As the aryloxy group for R³, preferred is an aryloxy group having 6-10 carbon atoms. Examples of the preferred aryloxy groups include phenoxy, 1-naphthoxy, and 2-naphthoxy. Particularly preferred is phenoxy group.

In the formula (1), each of p and q is preferably an integer of 0 to 2 (more preferably 0 or 1, and further preferably 0).

In the case where n is 1, r preferably is an integer of 1 to 3 (more preferably 1 or 2, and further preferably 1). In the case where r is 1, R³ is preferably placed at the center atom (meso-position) of the methine chain. Examples of the preferred R³ in that case include phenyl, methoxy, and phenoxy. Particularly preferred is phenyl group.

In the case where n is 0, r preferably is 1 or 2 (more preferably 1). In the case where r is 1, R³ is preferably placed at the center atom (meso-position) of the methine chain. Examples of the preferred R³ in that case include methyl, phenyl, and chlorine atom. Particularly preferred is phenyl group.

Next, the cation part is described below.

Examples of the onium ions represented by X^{k+} in the formula (1) include ammonium ion, oxonium ion, sulfonium ion, phosphonium ion, selenonium ion, and iodonium ion. Preferred is a quaternary ammonium ion.

The quaternary ammonium ion preferably used as X^{k+} can generally be prepared by alkylation (Menshutkin reaction), aralkylation, alkenylation, alkynylation, arylation, or hetero-cyclization of a tertiary amine (e.g., trimethylamine, triethylamine, tributylamine, triethanolamine, N-methylpyrrolidine, N-methylpiperidine, N,N-di-methylpiperazine, triethylenediamine, N,N,N′,N′-tetra-methylethylenediamine) or a nitrogen-containing heterocyclic ring (e.g., pyridine, picoline, 2,2′-dipyridyl, 4,4′-dipyridyl, 1,10-phenanthroline, quinoline, oxazole, thiazole, N-methylimidazole, pyrazine, tetrazole). The preferred X^{k+} is a quaternary ammonium ion comprising a nitrogen-containing heterocyclic ring, and particularly preferred is a quaternary pyridinium ion.

In the formula (1), k represents an integer of 1 to 10 (preferably 1 to 4, more preferably 2).

The particularly preferred X^{k+} is an onium ion of the following formula (2):

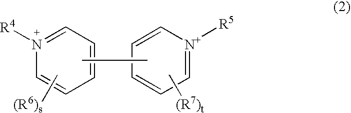

In the formula (2), each of R⁴ and R⁵ independently is an alkyl group, an alkenyl group, an alkynyl group, an aryl group, or a heterocyclic group. Each of R⁶ and R⁷ independently is a substituent group, and each of s and t independently represents an integer of 0 to 4. In the case where s or t is an integer of 2 or more, plural substituent groups represented by R⁶ or R⁷ may be the same or different or they may connect with each other to form a ring.

The ion of the formula (2) can be easily prepared by the known Menshutkin reaction [described in Japanese Patent Provisional Publication No. 61(1986)-148162] or the known arylation reaction [described in Japanese Patent Provisional Publications No. 51(1976)-16675 and No. 1(1989)-96171] between the corresponding dipyridyl ion and a halogen compound having the desired substituent group.

As the alkyl group for R⁴ or R⁵, preferred is an alkyl group having 1-18 (particularly 1-8) carbon atoms. The alkyl group may comprise a straight or branched chain or a cyclic structure. Examples of the alkyl groups include methyl, ethyl, n-propyl, isopropyl, n-butyl, sec-butyl, isobutyl, isoamyl, n-hexyl, cyclohexyl, 2-ethyl-hexyl, and n-octyl.

As the alkenyl group for R⁴ or R⁵, preferred is an alkenyl group having 2-18 (particularly 2-8) carbon atoms. Examples of the alkenyl groups include vinyl, 2-propenyl, 2-methylpropenyl, and 1,3-butadienyl.

As the alkynyl group for R⁴ or R⁵, preferred is an alkynyl group having 2-18 (particularly 2-8) carbon atoms. Examples of the alkynyl groups include ethynyl, propynyl, and 3,3-dimethylbutynyl.

As the aryl group for R⁴ or R⁵, preferred is an aryl group having 6-18 (particularly 6-10) carbon atoms. Examples of the aryl group include phenyl, 1-naphthyl, and 2-naphthyl.

As the heterocyclic group for R⁴ or R⁵, preferred is a saturated or unsaturated heterocyclic group having 4-7 carbon atoms. Preferred hetero-atoms contained in the heterocyclic group are nitrogen atom, oxygen atom, and sulfur atom. Examples of the preferred heterocyclic groups include 4-pyridyl, 2-pyridyl, 2-pyrazyl, 2-pyrimidyl, 2-imidazolyl, 2-furyl, 2-thiophenyl, 2-benzoxazolyl, and 2-benzothioxazolyl.

Each of R⁴ and R⁵ may have a substituent group. Examples of the substituent groups are the same as those described above for R¹ and R².

The substituent group represented by R⁶ or R⁷ in the formula (2) is the same as that of R¹ or R² in the formula (1), and is preferably an alkyl group having 1-18 carbon atoms. Particularly preferred is a unsubstituted alkyl group having 1-8 carbon atoms.

In the formula (2), each of s and t independently represents an integer of 0 to 2 (preferably 0 or 1, more preferably 0).

The two pyridine rings in the formula (2) may connect at any positions of the rings. Preferably, they link at 2- or 4-position (more preferably 4-position) of each ring.

Concrete examples of the oxonol compounds represented by the general formula (1) are given below, but they are by no means understood to restrict the invention.

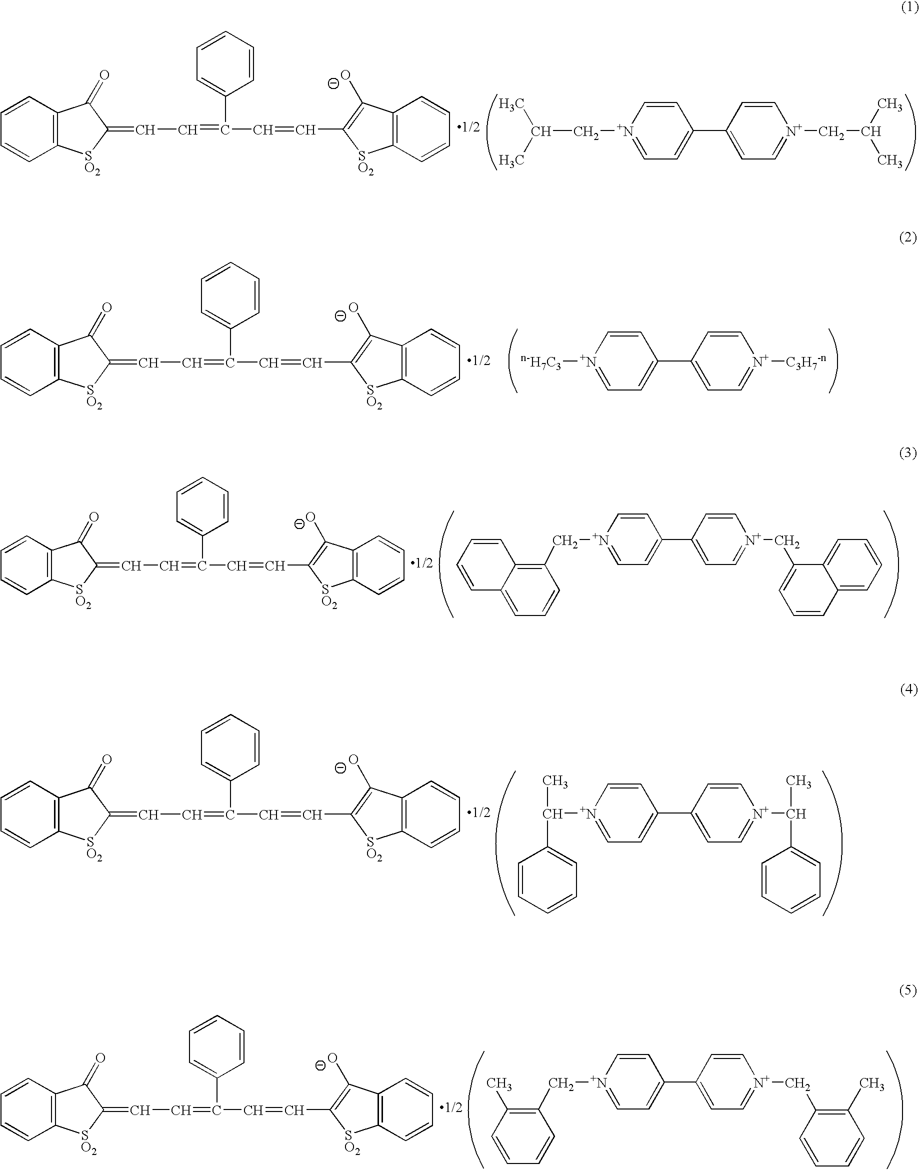

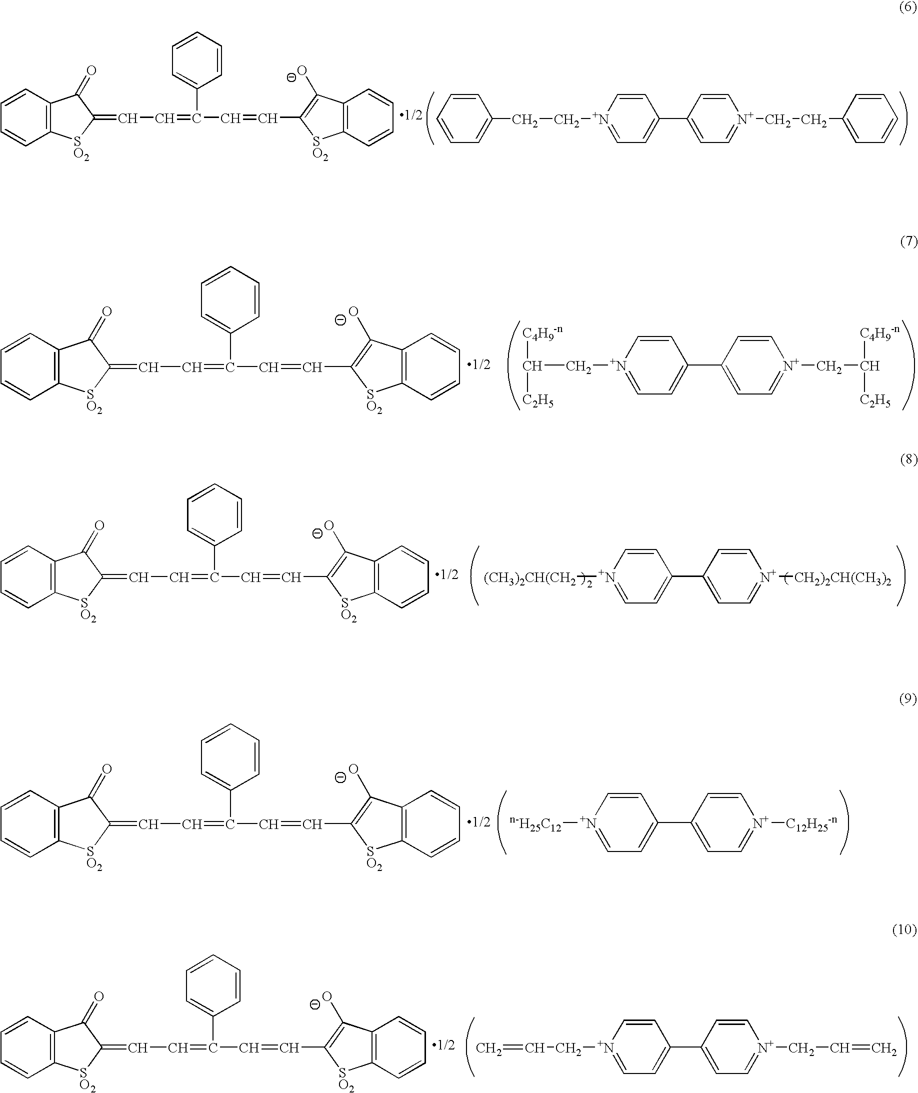

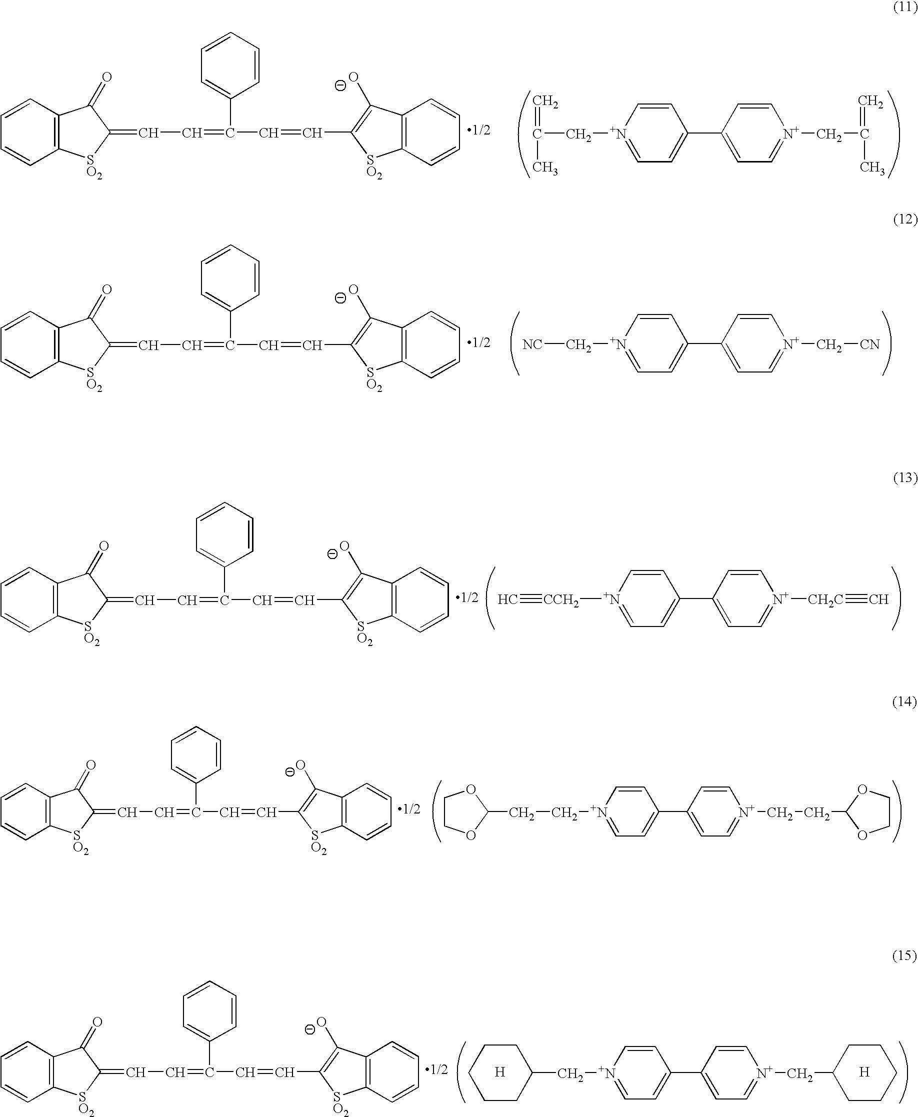

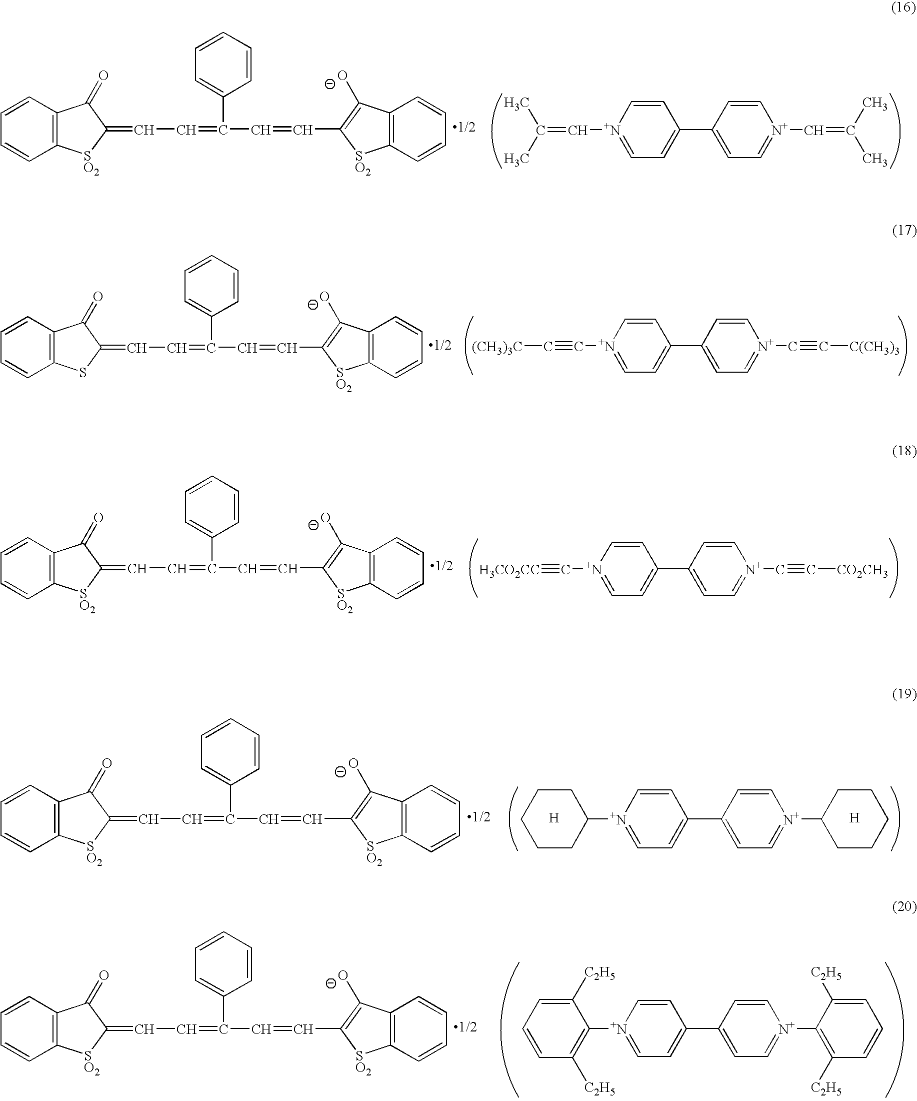

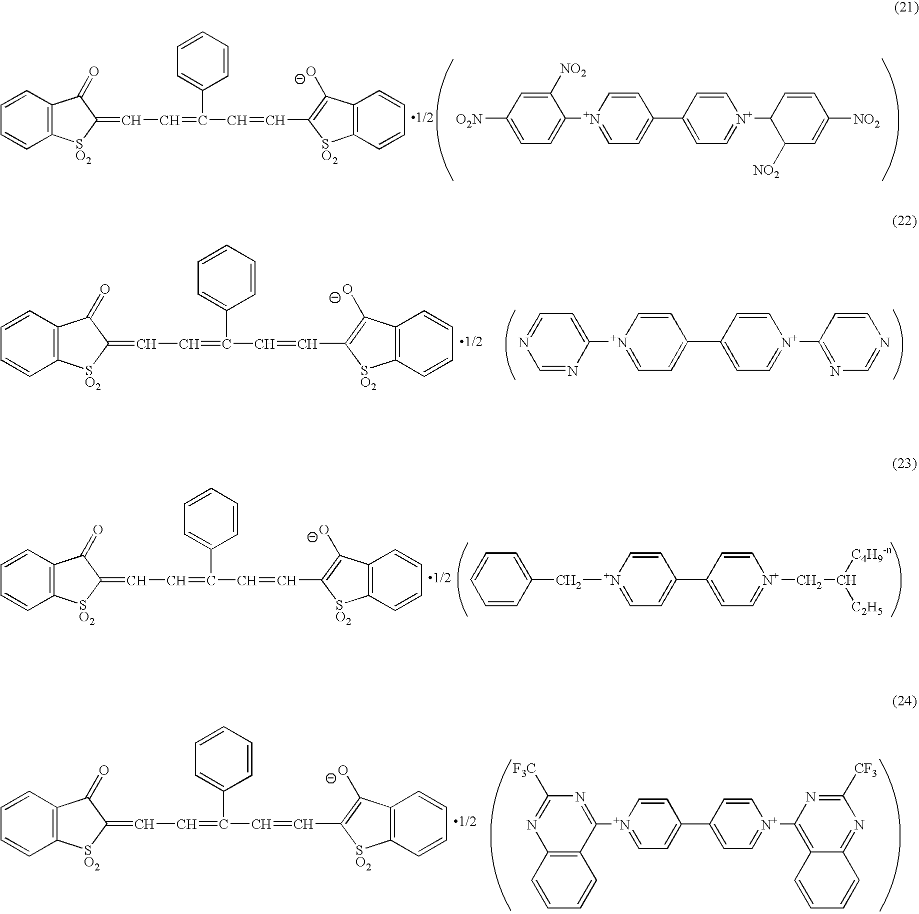

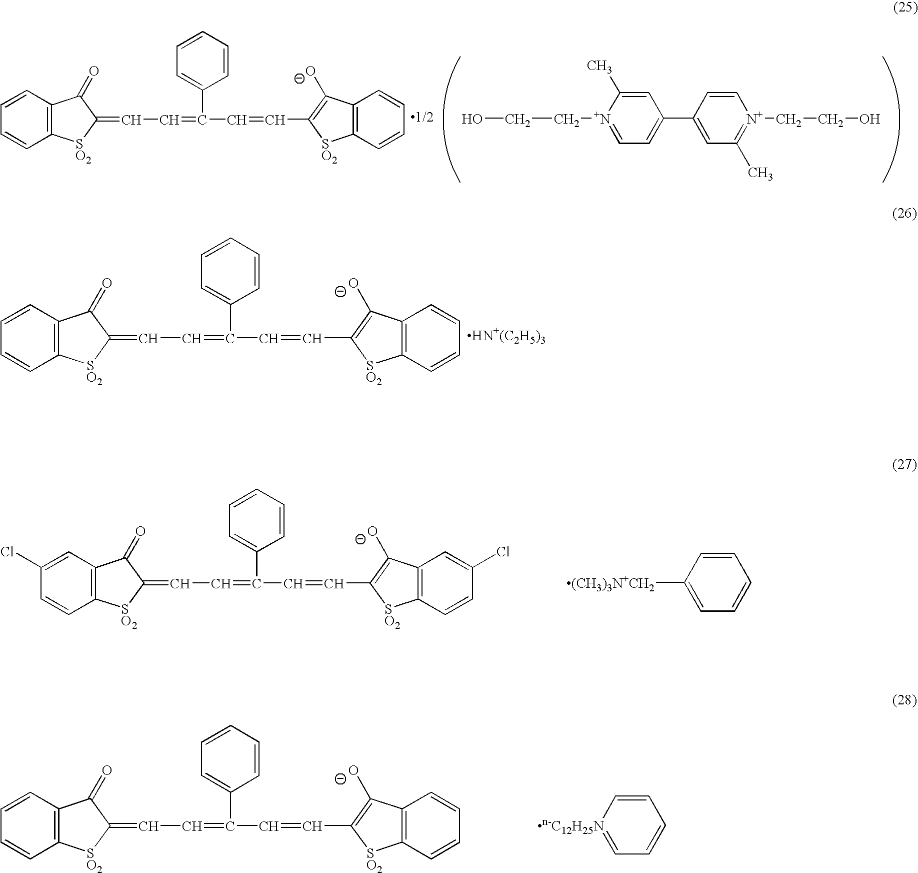

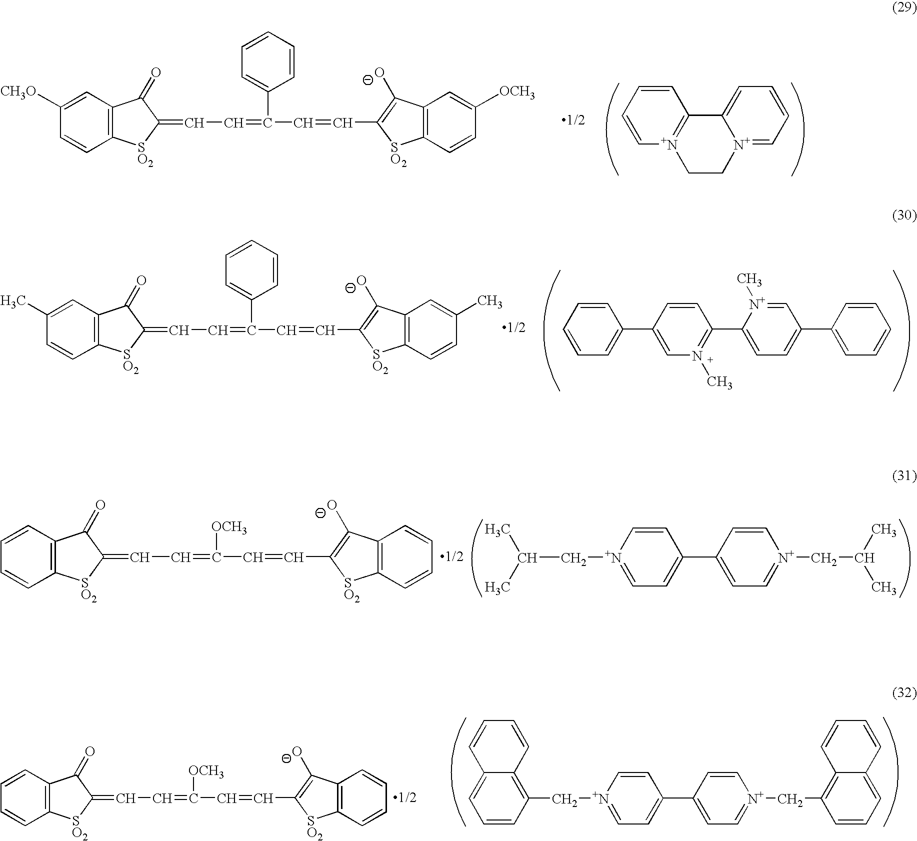

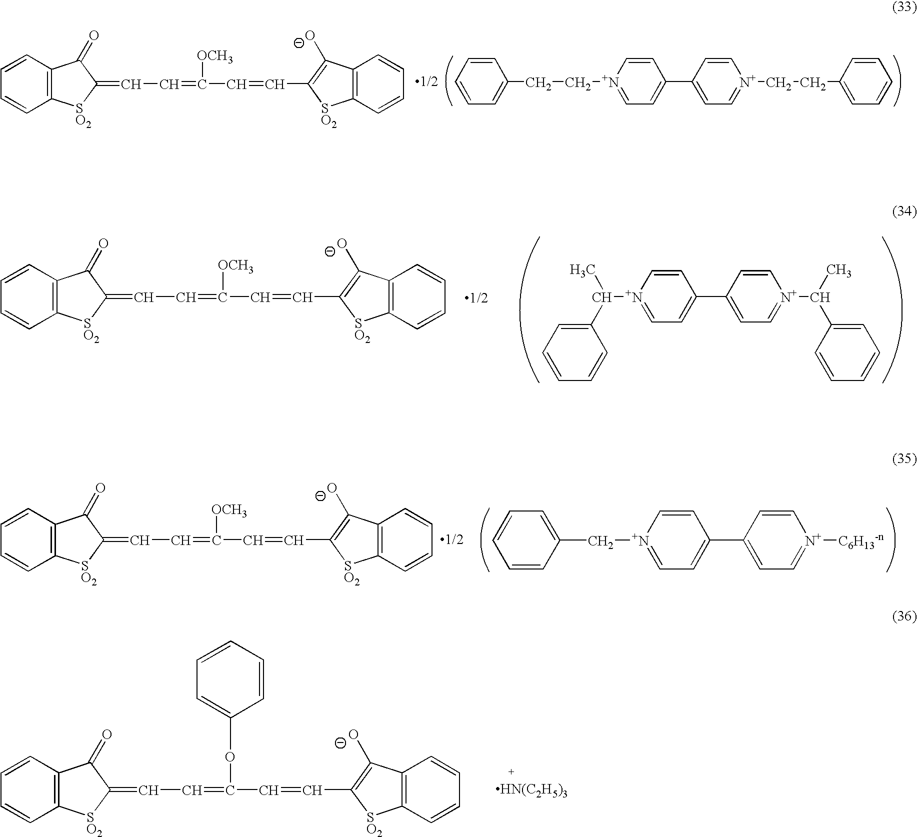

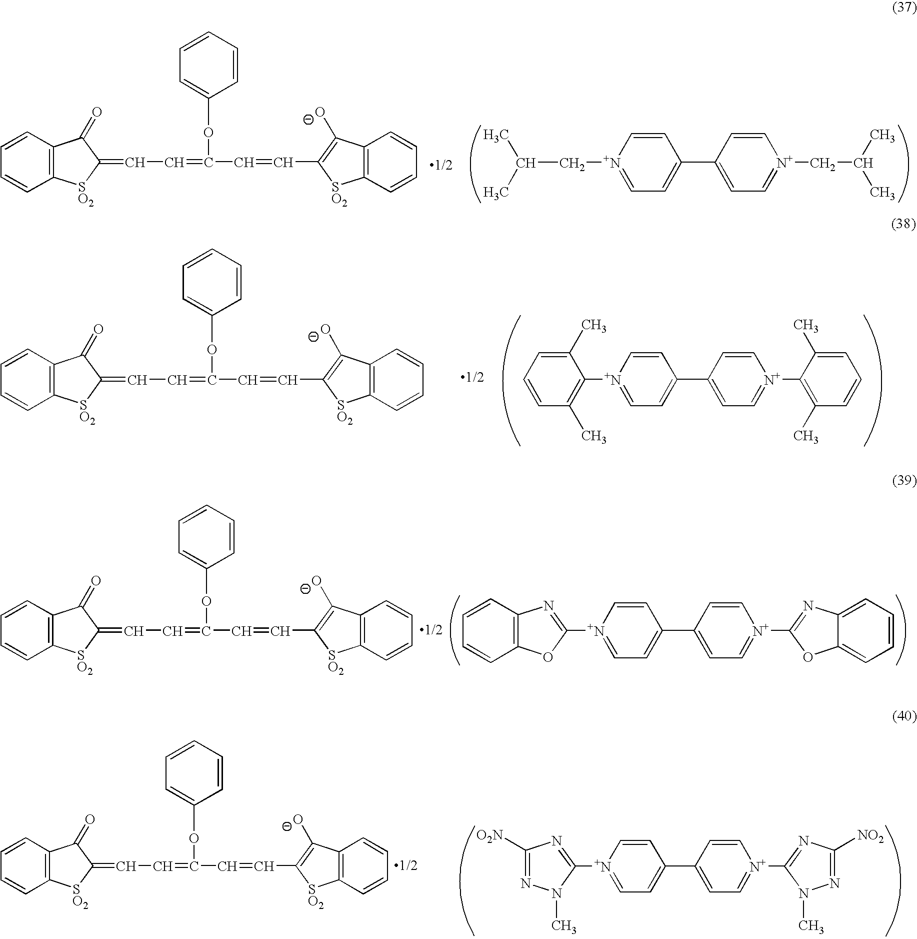

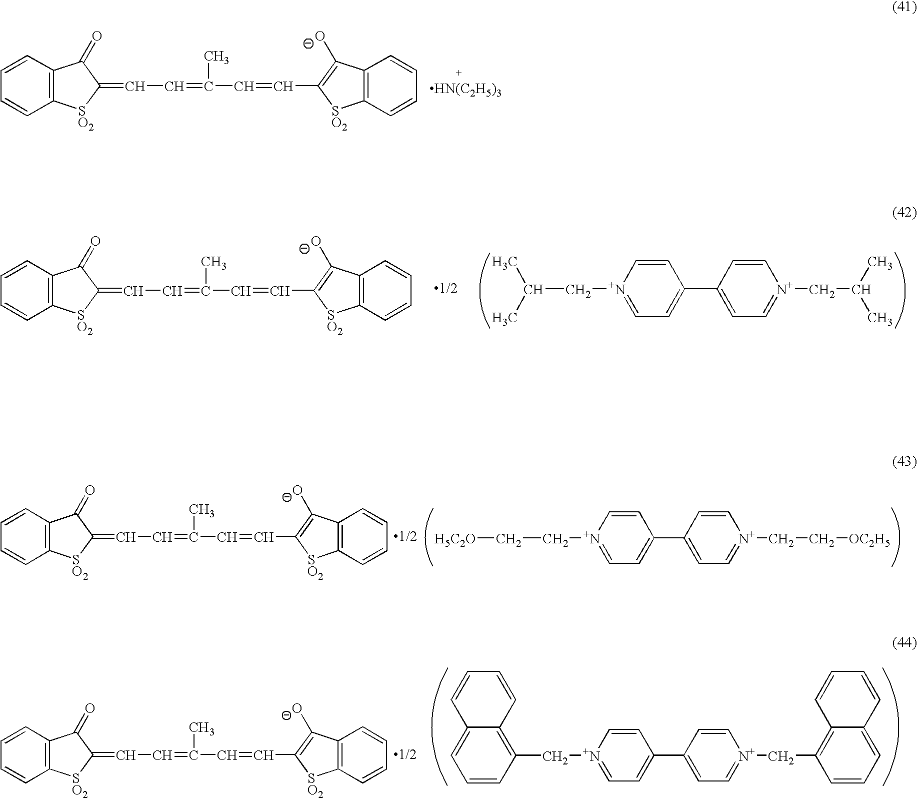

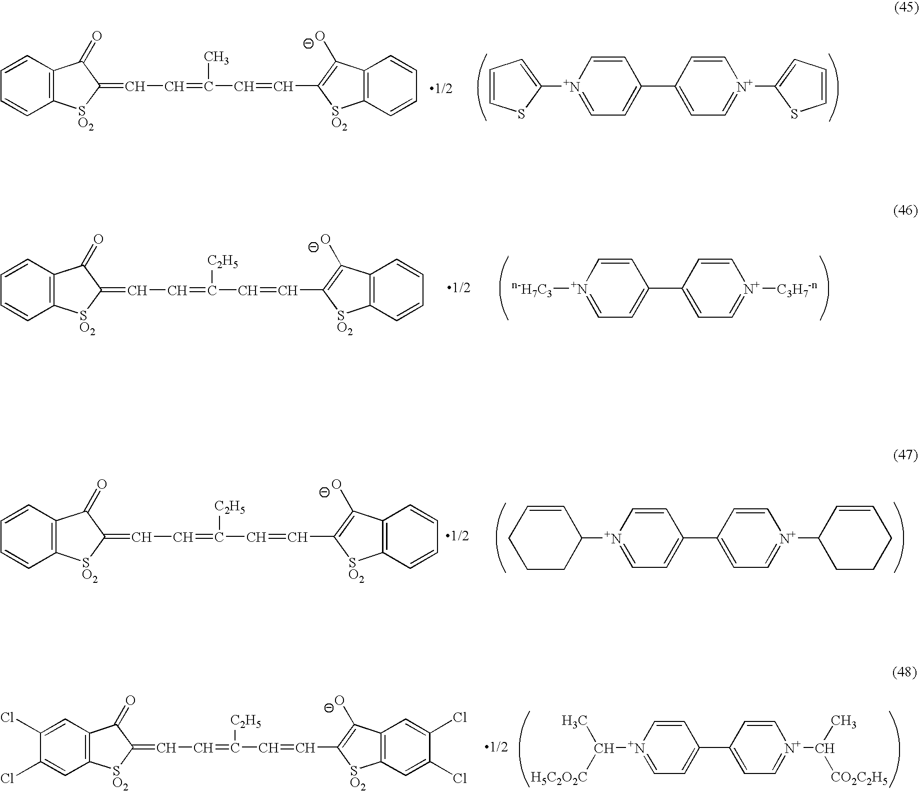

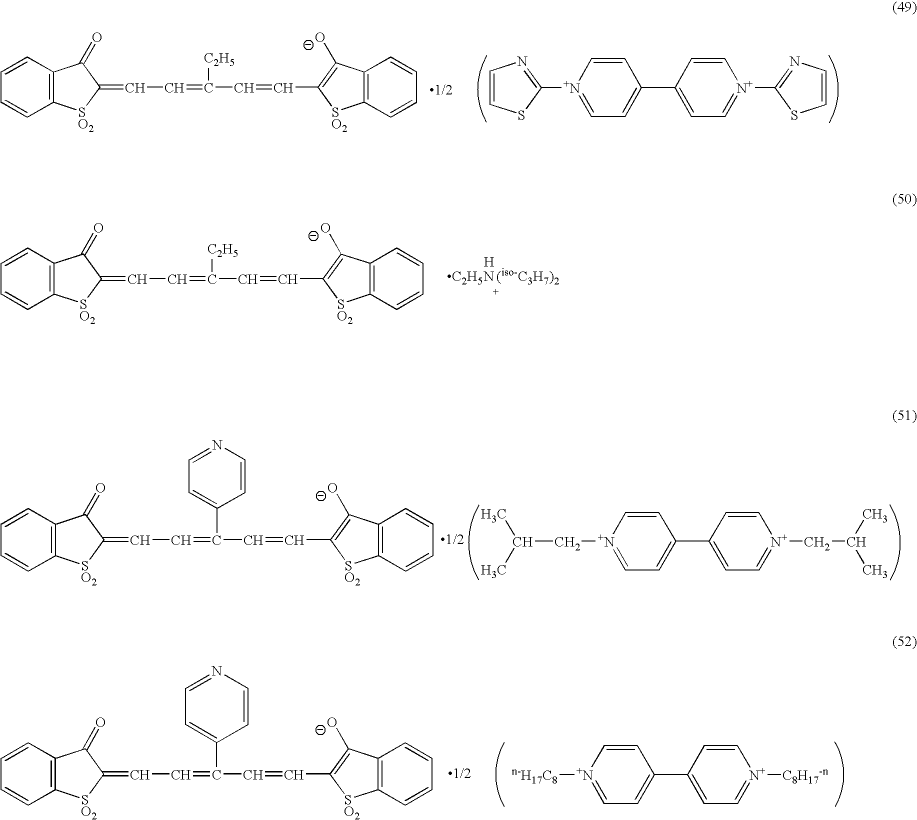

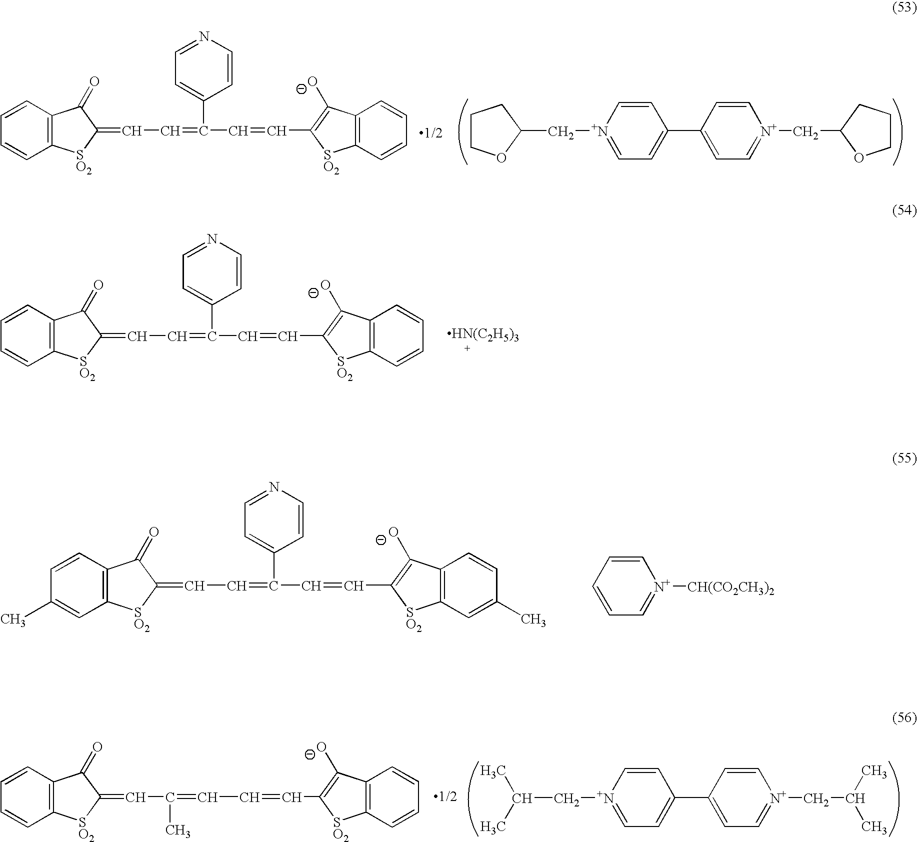

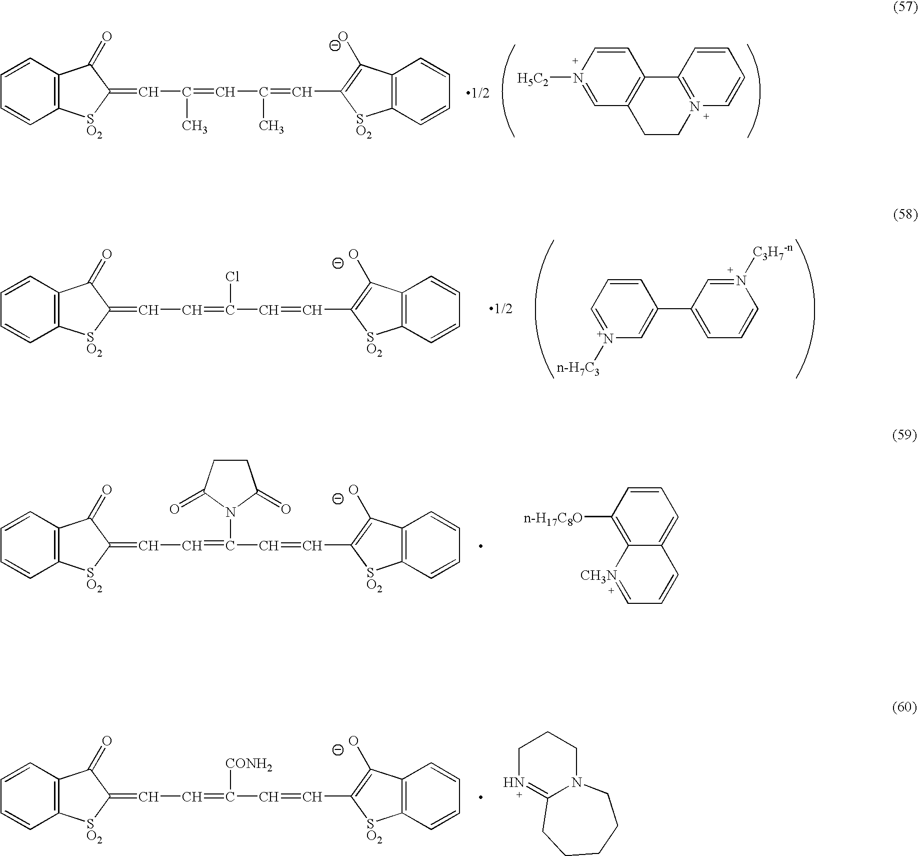

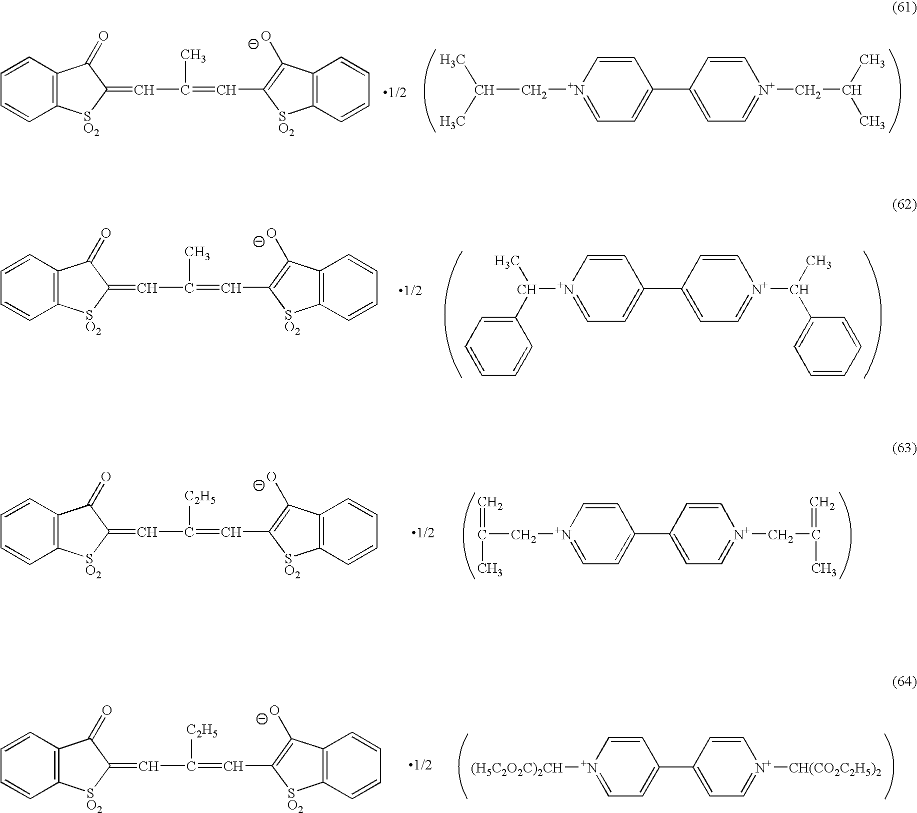

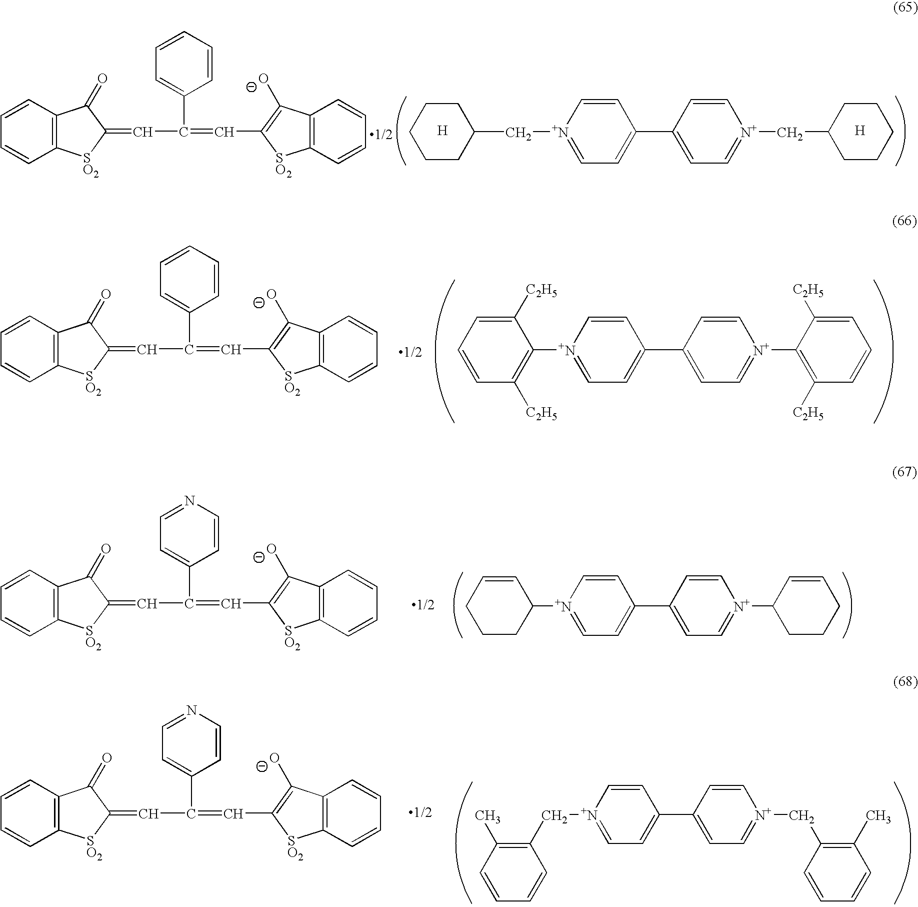

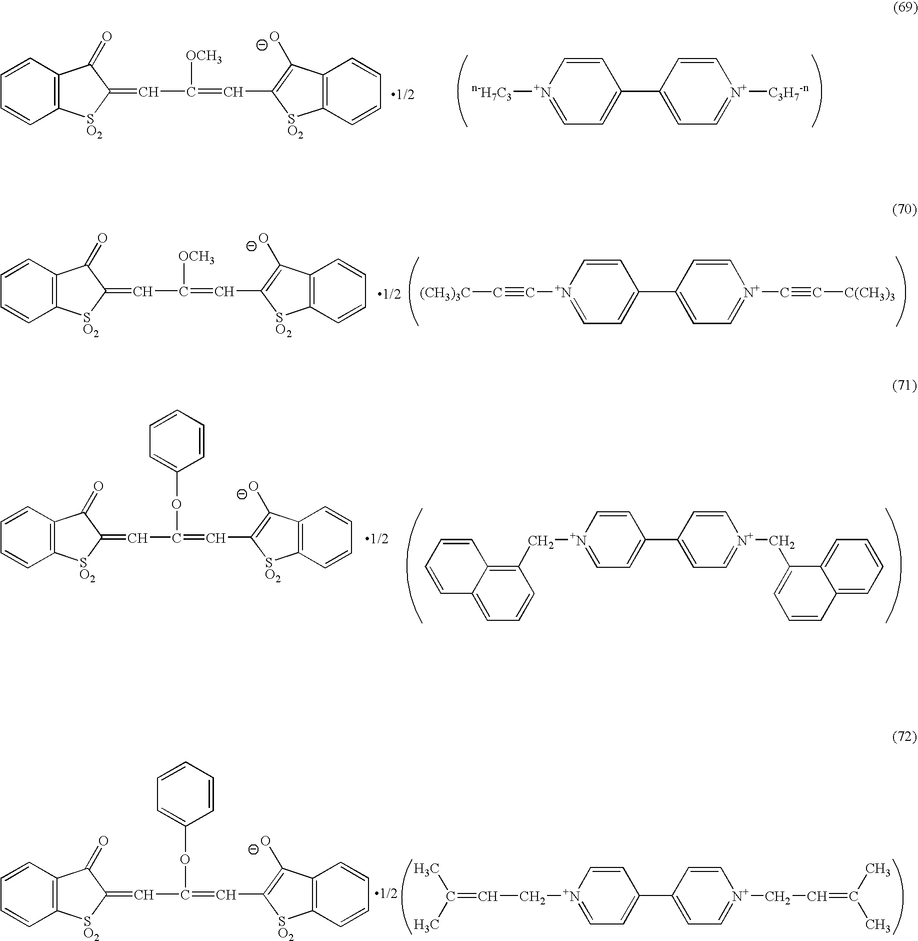

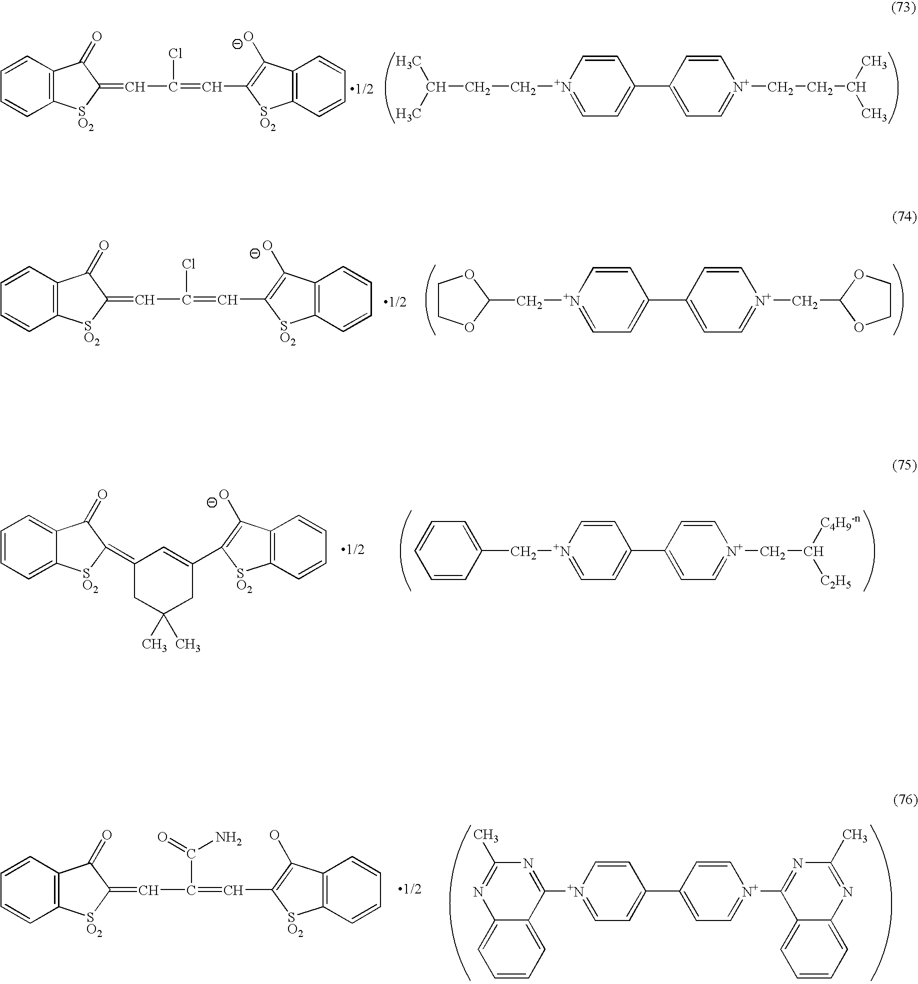

The oxonol compound of the general formula (1) can be prepared by the known counter-ion exchange reaction of the dye component (anion part), and the dye component can be synthesized by the known condensation reaction between the corresponding active methylene compound [3(2H)-thia-naphthenone-1,1-dioxide or its derivatives] and a methine source [a compound with which methine group is introduced]. Detailed descriptions of the methine sources are given in Japanese Patent Publications No. 39(1964)-22069, No. 43(1968)-3504, No. 52(1977)-38056, No. 54(1979)-38129, No. 55(1980)-10059, and No. 58 (1983)-35544; Japanese Patent Provisional Publications No. 49(1974)-99620, No. 52(1977)-92716, No. 59(1984)-16834, No. 63(1988)-316853, and No. 64(1989)-40827; U.K. Patent No. 1,133,986; and U.S. Pat. No. 3,247,127, No. 4,042,397, No. 4,181,225, No. 5,213,956, and No. 5,260,179.

Concrete examples of the methine sources are as follows. For introducing monomethine group, ortho-esters (e.g., ethyl orthoformate, ethyl orthoacetate) and N,N-diphenylformamidine hydrochloride are employable. The sources for trimethine group are, for example, tri-methoxypropane, 1,1,3,3-tetramethoxypropane, malon-aldehydedianyl hydrochloride, and their derivatives. Examples of the sources for the pentamethine group include glutaconaldehydedianyl hydrochloride, 1-(2,4-dinitrophenyl)-pyridinium chloride, and their derivatives.

Synthesis examples of the oxonol compounds represented by the general formula (1) are given below. In each compound mentioned below, the number in the parenthesis indicates that the compound has the formula of that number in the aforementioned compounds (1)-(76).

### SYNTHESIS EXAMPLE 1

### Preparation of Compound (1)

Compound (1) was prepared in accordance with the following scheme:

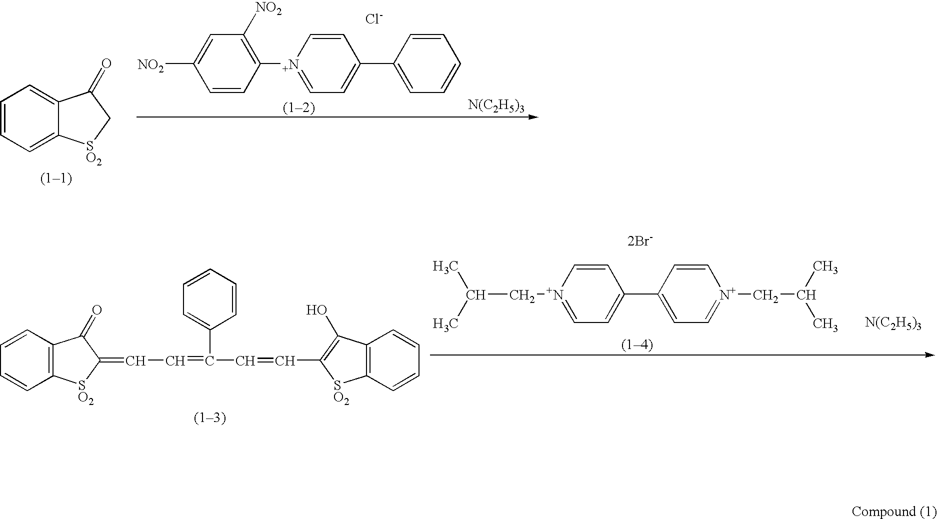

1) Preparation of Compound (1-3)

In 15 mL of N,N-dimethylformamide, 2.00 g of Compound (1-1) and 1.96 g of Compound (1-2) were dispersed. To the liquid stirred at room temperature, 3.33 g of triethylamine was added. The resulting mixture was stirred for 2 hours at room temperature, and then 80 mL of methanol and 2.74 mL of concentrated hydrochloric acid were successively added. The mixture was further stirred for 2 hours at room temperature, and the formed precipitate was collected, washed with methanol, and dried to obtain 2.50 g of Compound (1-3) in the form of a dark green needle crystalline product.

2) Preparation of Compound (1)

In 15 mL of methanol, 200 mg of Compound (1-3) and 85.6 mg of Compound (1-4) were dispersed. To the liquid stirred at room temperature, 40.3 mg of triethylamine was added. The resulting mixture was stirred for 2 hours at room temperature, and the formed precipitate was collected, washed with methanol, and dried to obtain 230 mg of Compound (1) in the form of a green crystalline product.

¹H-NMR (DMSO-d₆): 9.35 (2H, d), 8.79 (2H, d), 7.95 (2H, d), 7.70-7.88 (6H, m), 7.50-7.65 (3H, m), 7.35 (2H, dd), 6.94 (4H, bs), 4.54 (2H, d), 2.30 (1H, tq), 0.95 (6H, d).

### SYNTHESIS EXAMPLE 2

### Preparation of Compound (4)

Compound (4) was prepared in accordance with the following scheme:

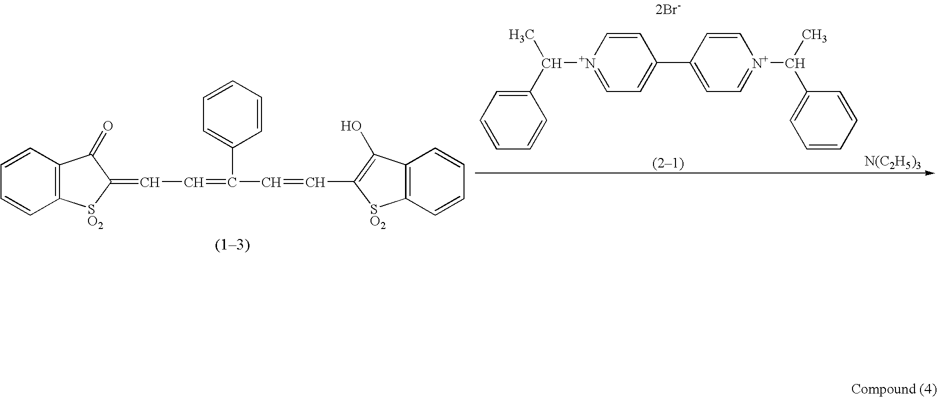

In 15 mL of methanol, 200 mg of Compound (1-3) and 105 mg of Compound (2-1) were dispersed. To the liquid stirred at room temperature, 40.3 mg of triethylamine was added. The resulting mixture was stirred for 2 hours at room temperature, and the formed precipitate was collected, washed with methanol, and dried to obtain 160 mg of Compound (4) in the form of a green crystalline product.

¹H-NMR (DMSO-d₆): 9.55 (2H, d), 8.70 (2H, d), 8.05-7.25 (17H, m), 6.95 (2H, bs), 6.70 (2H, bs), 6.32 (1H, dd), 4.12 (1H, q), 2.11 (3H, d).

### SYNTHESIS EXAMPLE 3

### Preparation of Compound (7)

Compound (7) was prepared in accordance with the following scheme:

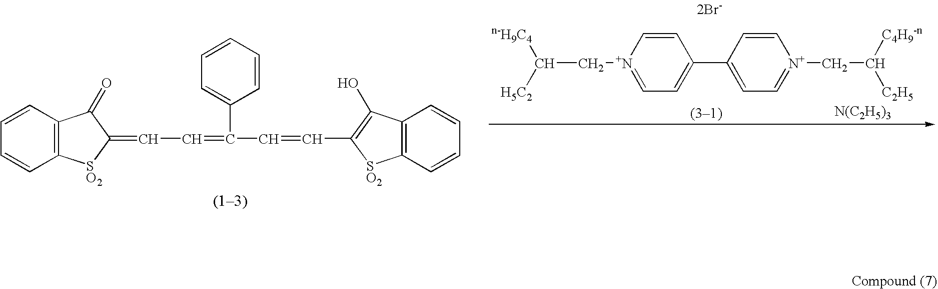

In 15 mL of methanol, 200 mg of Compound (1-3) and 105 mg of Compound (3-1) were dispersed. To the liquid stirred at room temperature, 40.3 mg of triethylamine was added. The resulting mixture was stirred for 2 hours at room temperature, and the formed precipitate was collected, washed with methanol, and dried to obtain 210 mg of Compound (7) in the form of a dark green crystalline product.

¹H-NMR (DMSO-d₆): 9.35 (2H, d), 8.80 (2H, d), 7.99 (2H, d), 7.80 (6H, bs), 7.60 (3H, s), 7.35 (2H, s), 6.95 (2H, bs), 6.69 (2H, bd), 4.63 (2H, bs), 2.09 (1H, m), 1.15-1.46 (8H, m), 0.95-0.80 (6H, m).

### SYNTHESIS EXAMPLE 4

### Preparation of Compound (8)

Compound (8) was prepared in accordance with the following scheme:

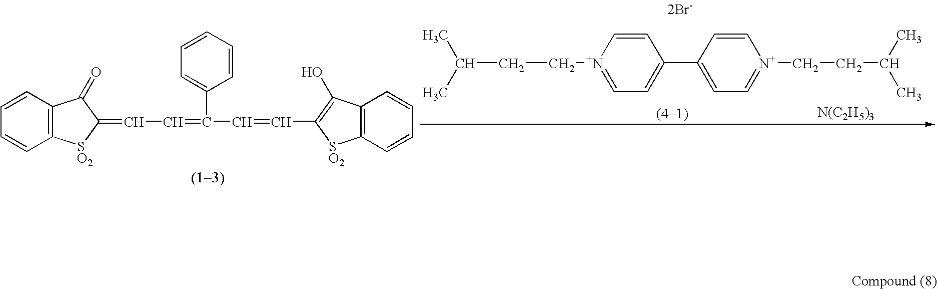

In 30 mL of methanol, 1.5 g of Compound (1-3) and 0.623 g of Compound (4-1) were dispersed. To the liquid stirred at room temperature, 0.275 g of triethylamine was added. The resulting mixture was stirred for 1 hour at room temperature, and the formed precipitate was collected, washed with methanol, and dried to obtain 1.46 g of Compound (8) in the form of a dark green columnar crystalline product.

¹H-NMR (DMSO-d₆): 9.40 (2H, d), 8.77 (2H, d), 7.99 (2H, d), 7.80 (6H, bs), 7.60 (3H, bs), 7.36 (2H, bs), 6.95 (2H, bd), 6.69 (2H, bd), 4.70 (2H, t), 1.90 (2H, dt), 1.65 (1H, tq), 1.00 (6H, d).

### SYNTHESIS EXAMPLE 5

### Preparation of Compound (11)

Compound (11) was prepared in accordance with the following scheme:

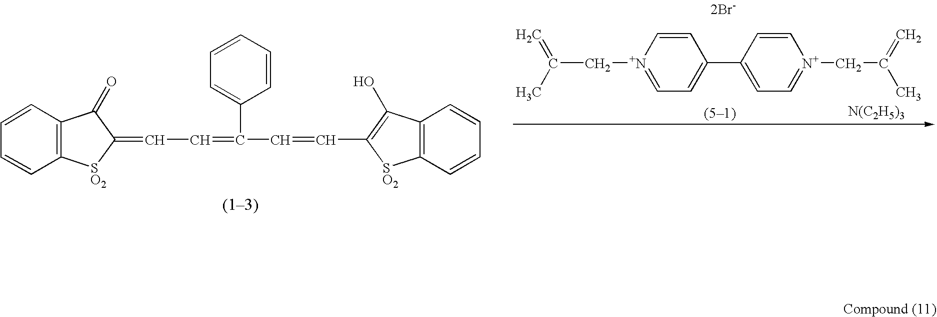

In 50 mL of methanol, 1.00 g of Compound (1-3) and 0.306 g of Compound (5-1) were dispersed. To the liquid stirred at room temperature, 0.234 g of triethylamine was added. The resulting mixture was stirred for 1.5 hours at room temperature, and the formed precipitate was collected, washed with methanol, and dried to obtain 0.950 g of Compound (11) in the form of a golden crystalline product.

¹H-NMR (DMSO-d₆): 9.31 (2H, d), 8.80 (2H, d), 7.99 (2H, d), 7.95-7.75 (6H, m), 7.60 (3H, bs), 7.35 (2H, bs), 6.96 (2H, bd), 6.68 (2H, d), 5.34 (2H, s), 5.20 (1H, s), 4.90 (1H, s), 1.89 (3H, s).

### SYNTHESIS EXAMPLE 6

### Preparation of Compound (26)

In 30 mL of methanol, 0.60 g of Compound (1-3) was dispersed. To the liquid stirred at room temperature, 0.11 g of triethylamine was added. The resulting mixture was stirred for 1.5 hours at room temperature, and further stirred for 1 hour, while the liquid was chilled in ice-cold water. The formed precipitate was collected, washed with cold methanol, and dried to obtain 0.57 g of Compound (26) in the form of a dark purple crystalline product.

¹H-NMR (DMSO-d₆): 8.85 (1H, bs), 7.75 (2H, bd), 7.90-7.71 (6H, m), 7.60 (3H, m), 7.35 (2H, m), 6.96 (2H, bs), 6.70 (2H, bd), 3.15-3.04 (6H, m), 1.20 (9H, t).

### SYNTHESIS EXAMPLE 7

### Preparation of Compound (31)

Compound (31) was prepared in accordance with the following scheme:

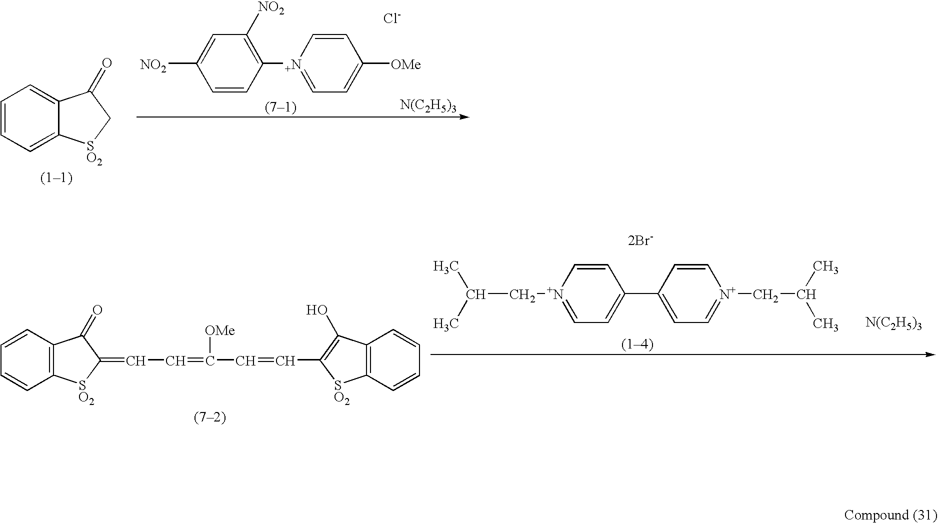

1) Preparation of Compound (6-2)

In 12 mL of N,N-dimethylformamide, 1.46 g of Compound (1-1) and 1.25 g of Compound (7-1) were dispersed. To the liquid stirred at room temperature, 1.69 mL of triethylamine was added. The resulting mixture was stirred for 2 hours at room temperature, and then 60 mL of methanol and 1.0 mL of concentrated hydrochloric acid were successively added. The mixture was further stirred for 2 hours at room temperature, and the formed precipitate was collected, washed with methanol, and dried to obtain 1.75 g of Compound (7-2) in the form of a black powder.

2) Preparation of Compound (31)

In 20 mL of methanol, 0.23 g of Compound (7-2) and 0.13 g of Compound (1-4) were dispersed. To the liquid stirred at room temperature, 0.07 mL of triethylamine was added. The resulting mixture was stirred for 1.5 hours at room temperature, and the formed precipitate was collected, washed with methanol, and dried to obtain 0.20 g of Compound (31) in the form of a dark blue crystalline product.

¹H-NMR (DMSO-d₆): 9.35 (2H, d), 8.80 (2H, d), 7.99 (2H, d), 7.90-7.75 (3H, m), 7.30 (2H, d), 7.18 (2H, d), 7.05 (1H, bd), 6.67 (2H, d), 4.55 (2H, d), 3.90 (3H, s), 2.30 (1H, tq), 0.95 (6H, d).

### SYNTHESIS EXAMPLE 8

### Preparation of Compound (32)

Compound (32) was prepared in accordance with the following scheme:

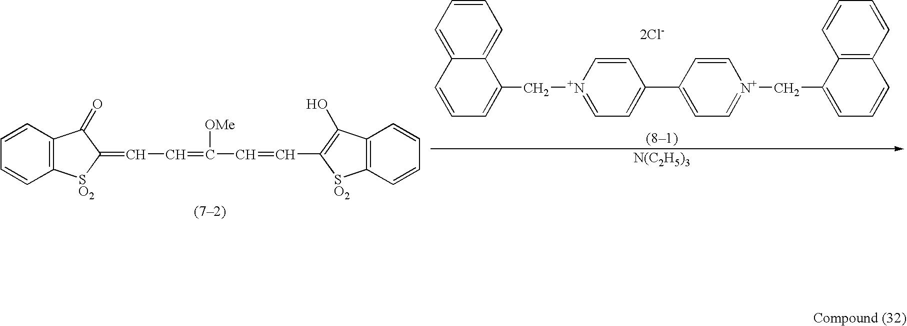

In 20 mL of methanol, 0.23 g of Compound (7-2) and 0.17 g of Compound (8-1) were dispersed. To the liquid stirred at room temperature, 0.07 mL of triethylamine was added. The resulting mixture was stirred for 1.5 hours at room temperature, and the formed precipitate was collected, washed with methanol, and dried to obtain 0.21 g of Compound (31) in the form of a dark blue crystalline product.

¹H-NMR (DMSO-d₆): 9.45 (2H, d), 8.70 (2H, d), 8.25-7.45 (9H, m), 7.29 (2H, d), 7.16 (2H, d), 7.05 (2H, bs), 7.70 (2H, bs), 6.50 (2H, s), 3.90 (3H, s).

### SYNTHESIS EXAMPLE 9

### Preparation of Compound (36)

Compound (36) was prepared in accordance with the following scheme:

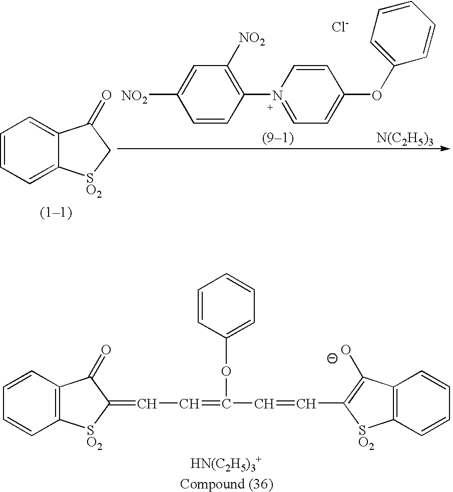

In 24 mL of N,N-dimethylformamide, 2.9 g of Compound (1-1) and 2.3 g of Compound (9-1) were dispersed. To the liquid stirred at room temperature, 3.4 mL of triethylamine was added. The resulting mixture was stirred for 4 hours at room temperature, and then 80 mL of methanol and 2.0 mL of concentrated hydrochloric acid were successively added. The mixture was further stirred for 1.5 hours at room temperature, and the formed precipitate was collected. The precipitate was then dispersed in 80 mL of methanol, and 3.0 mL of triethylamine was added, while the liquid was stirred at room temperature. After the dispersion was left overnight, the precipitate was collected, washed with methanol, and dried to obtain 1.1 g of Compound (36) in the form of a green crystalline product.

¹H-NMR (DMSO-d₆): 8.80 (1H, bs), 8.00-7.10 (15H, m), 6.30 (2H, bd), 3.10-3.00 (6H, m), 1.20 (9H, t).

### SYNTHESIS EXAMPLE 10

### Preparation of Compound (37)

In 20 mL of methanol, 0.23 g of Compound (36) and 0.13 g of Compound (1-4) were dispersed. The resulting mixture was stirred for 1.5 hours at room temperature, and the formed precipitate was collected, washed with methanol, and dried to obtain 0.20 g of Compound (37) in the form of a dark blue crystalline product.

¹H-NMR (DMSO-d₆): 9.35 (2H, d), 8.78 (2H, d), 8.00-7.10 (15H, m), 6.26 (2H, bd), 4.55 (2H, d), 2.30 (1H, tq), 0.95 (6H, t).

### SYNTHESIS EXAMPLE 11

### Preparation of Compound (41)

Compound (41) was prepared in accordance with the following scheme:

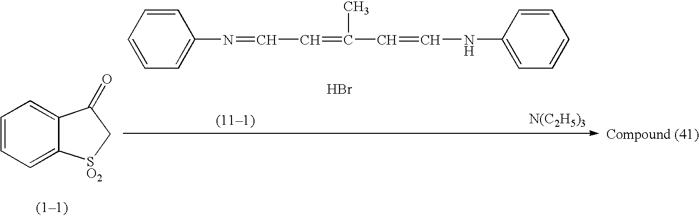

In 30 mL of N,N-dimethylformamide, 3.64 g of Compound (1-1) and 3.43 g of Compound (11-1) were dispersed. To the liquid stirred at room temperature, 4.22 g of triethylamine was added. The resulting mixture was stirred for 4 hours at room temperature, and then 200 mL of water was added. The formed precipitate was collected, washed with methanol, and dried to obtain 3.10 g of Compound, (41) in the form of a green crystalline product.

¹H-NMR (DMSO-d₆): 8.79 (1H, bs), 8.05-7.35 (10H, m), 7.50 (2H, bd), 3.16-3.30 (6H, m), 2.30 (3H, s), 1.21 (9H, t).

### SYNTHESIS EXAMPLE 12

### Preparation of Compound (44)

In 20 mL of methanol, 0.27 g of Compound (41) and 0.16 g of Compound (8-1) were dispersed. The resulting mixture was stirred for 1.5 hours at room temperature, and the formed precipitate was collected, washed with methanol, and dried to obtain 0.19 g of Compound (44) in the form of a dark green crystalline product.

¹H-NMR (DMSO-d₆): 9.43 (2H, d), 8.70 (2H, d), 8.22-7.40 (24H, m), 6.55 (2H, bs), 6.47 (2H, s), 2.29 (3H, s).

### SYNTHESIS EXAMPLE 13

### Preparation of Compound (46)

Compound (46) was prepared in accordance with the following scheme:

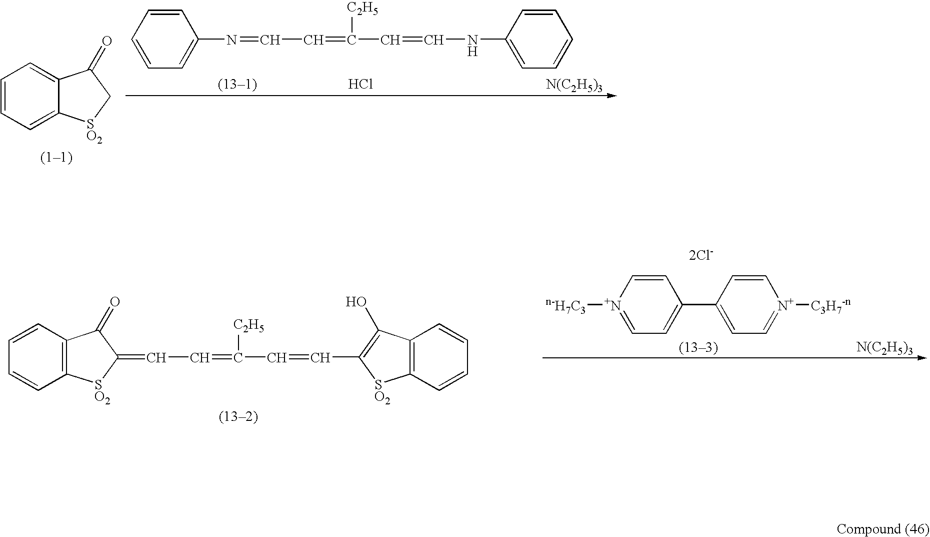

1) Preparation of Compound (13-2)

In 15 mL of N,N-dimethylformamide, 874 mg of Compound (1-1) and 750 mg of Compound (13-1) were dispersed. To the liquid stirred at room temperature, 2.00 mL of triethylamine was added. The resulting mixture was stirred for 3 hours at room temperature, and then 80 mL of methanol and 1.2 mL of concentrated hydrochloric acid were successively added. The mixture was further stirred for 1.5 hours, while the liquid was chilled in ice-cold water, and the formed precipitate was collected, washed with methanol, and dried to obtain 640 mg of Compound (13-2) in the form of a blue crystalline product.

2) Preparation of Compound (46)

In 20 mL of N,N-dimethylformamide, 200 mg of Compound (13-2) and 88.5 mg of Compound (13-3) were dispersed. To the liquid stirred at room temperature, 44.5 g of triethylamine was added. The resulting mixture was stirred for 2 hours at room temperature, and the formed precipitate was collected, washed with methanol, and dried to obtain 220 mg of Compound (46) in the form of a golden needle crystalline product.

¹H-NMR (DMSO-d₆): 9.35 (2H, d), 8.76 (2H, d), 8.08-7.20 (10H, m), 6.40 (2H, d), 4.65 (2H, t), 2.80 (2H, q), 2.00 (2H, tq), 1.25 (3H, t), 0.95 (3H, t).

### SYNTHESIS EXAMPLE 14

### Preparation of Compound (51)

Compound (51) was prepared in accordance with the following scheme:

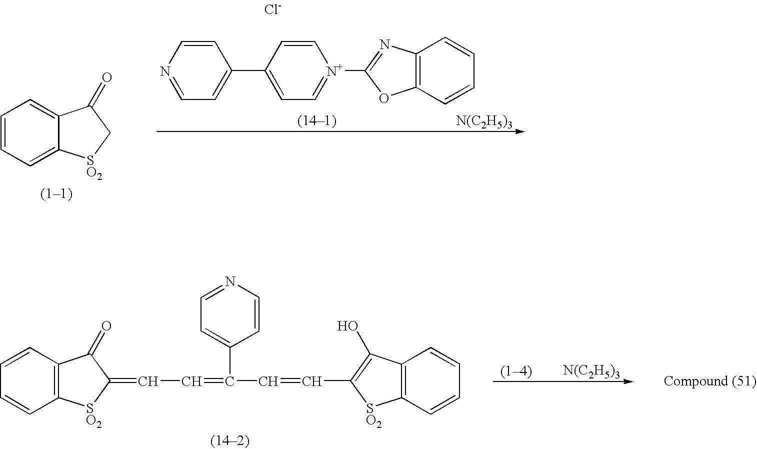

1) Preparation of Compound (14-2)

In 30 mL of N,N-dimethylformamide, 2.00 g of Compound (1-1) and 1.70 g of Compound (14-1) were dispersed. To the liquid stirred at room temperature, 4.59 mL of triethylamine was added. The resulting mixture was stirred for 4 hours at room temperature, and then 100 mL of methanol and 2.74 mL of concentrated hydrochloric acid were successively added. The mixture was further stirred for 1.5 hours, while the liquid was chilled in ice-cold water, and the formed precipitate was collected, washed with methanol, and dried to obtain 2.11 g of Compound (14-2) in the form of a dark blue crystalline product.

2) Preparation of Compound (51)

In 15 mL of N,N-dimethylformamide, 200 mg of Compound (14-2) and 79.6 mg of Compound (1-4) were dispersed. To the liquid stirred at room temperature, 40.3 mg of triethylamine was added. The resulting mixture was stirred for 2 hours at room temperature, and the formed precipitate was collected, washed with methanol, and dried to obtain 220 mg of Compound (46) in the form of a golden needle crystalline product.

¹H-NMR (DMSO-d₆): 9.34 (2H, d), 8.75 (4H, m), 7.97 (2H, d), 7.90-7.75 (6H, m), 7.44 (2H, d), 6.70 (2H, bd), 6.56 (2H, d), 4.53 (2H, d), 2.28 (1H, tq), 0.93 (6H, d).

### SYNTHESIS EXAMPLE 15

### Preparation of Compound (61)

Compound (61) was prepared in accordance with the following scheme:

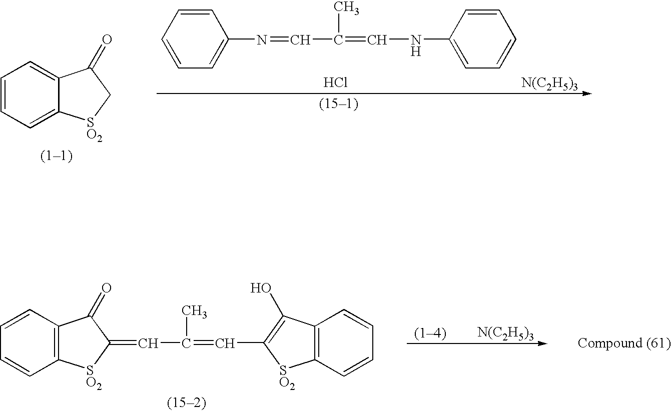

1) Preparation of Compound (15-2)

In 30 mL of N,N-dimethylformamide, 2.00 g of Compound (1-1) and 1.50 g of Compound (15-1) were dispersed. To the liquid stirred at room temperature, 4.59 mL of triethylamine was added. The mixture was stirred for 2.5 hours at room temperature, and then 150 mL of methanol and 3 mL of concentrated hydrochloric acid were successively added. The mixture was further stirred for 2 hours at room temperature, and the formed precipitate was collected, washed with methanol, and dried to obtain 2.10 g of Compound (15-2) in the form of a dark green crystalline product.

2) Preparation of Compound (61)

In 20 mL of methanol, 200 mg of Compound (15-2) and 104 mg of Compound (1-4) were dispersed. To the liquid stirred at room temperature, 48.8 mg of triethylamine was added. The resulting mixture was stirred for 2 hours at room temperature, and the formed precipitate was collected, washed with methanol, and dried to obtain 180 mg of Compound (61) in the form of a dark green crystalline product.

¹H-NMR (DMSO-d₆): 9.39 (2H, d), 8.83 (4H, m), 8.00-7.80 (8H, m), 7.53 (2H, s), 4.53 (2H, d), 2.57 (3H, s), 2.29 (1H, tq), 0.93 (6H, d).

### SYNTHESIS EXAMPLE 16

### Preparation of Compound (63)

Compound (63) was prepared in accordance with the following scheme:

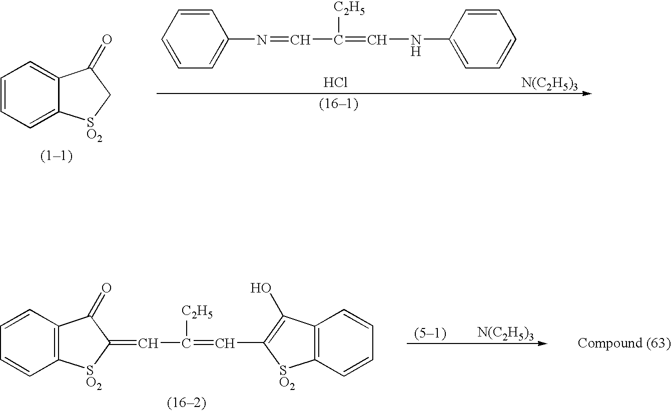

1) Preparation of Compound (16-2)

In 30 mL of N,N-dimethylformamide, 2.00 g of Compound (1-1) and 1.57 g of Compound (16-1) were dispersed. To the liquid stirred at room temperature, 4.59 mL of triethylamine was added. The mixture was stirred for 2 hours at room temperature, and then 150 mL of methanol and 3 mL of concentrated hydrochloric acid were successively added. The mixture was further stirred for 2 hours at room temperature, and the formed precipitate was collected, washed with methanol, and dried to obtain 2.05 g of Compound (16-2) in the form of a dark green crystalline product.

2) Preparation of Compound (63)

In 20 mL of methanol, 200 mg of Compound (16-2) and 78.7 mg of Compound (5-1) were dispersed. To the liquid stirred at room temperature, 47.2 mg of triethylamine was added. The resulting mixture was stirred for 2 hours at room temperature, and the formed precipitate was collected, washed with methanol, and dried to obtain 180 mg of Compound (63) in the form of a dark green crystalline product.

¹H-NMR (DMSO-d₆): 9.37 (2H, d), 8.84 (2H, d), 8.01-7.78 (8H, m), 7.52 (2H, s), 5.34 (2H, s), 5.20 (1H, s), 4.92 (1H, s), 2.79 (2H, q), 1.76 (3H, s), 1.21 (3H, t).

### SYNTHESIS EXAMPLE 17

### Preparation of Compound (65)

Compound (65) was prepared in accordance with the following scheme:

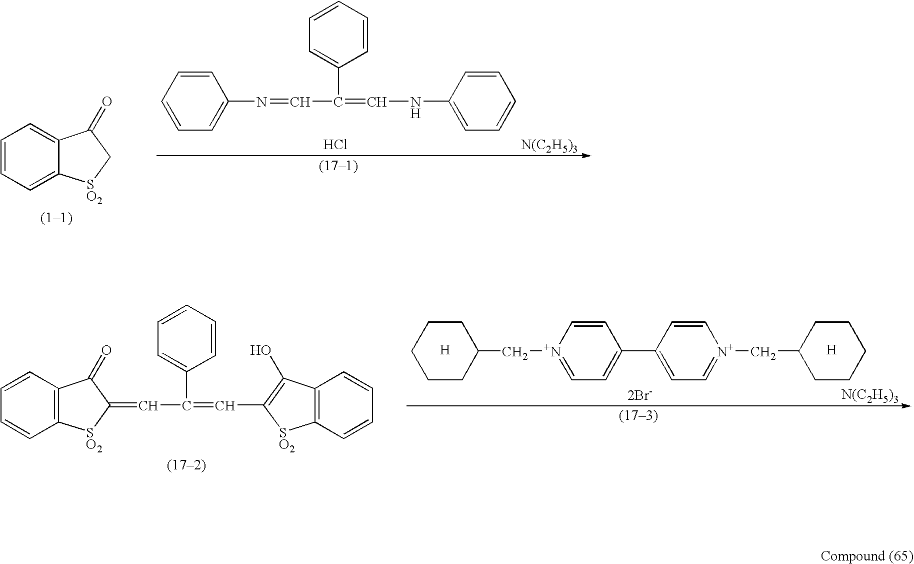

1) Preparation of Compound (17-2)

In 30 mL of N,N-dimethylformamide, 2.00 g of Compound (1-1) and 1.84 g of Compound (17-1) were dispersed. To the liquid stirred at room temperature, 4.59 mL of triethylamine was added. The mixture was stirred for 2.5 hours at room temperature, and then 150 mL of methanol and 3 mL of concentrated hydrochloric acid were successively added. The mixture was further stirred for 2 hours at room temperature, and the formed precipitate was collected, washed with methanol, and dried to obtain 1.98 g of Compound (17-2) in the form of a dark green crystalline product.

2) Preparation of Compound (65)

In 20 mL of methanol, 200 mg of Compound (17-2) and 107 mg of Compound (17-3) were dispersed. To the liquid stirred at room temperature, 42.5 mg of triethylamine was added. The resulting mixture was stirred for 2 hours at room temperature, and the formed precipitate was collected, washed with methanol, and dried to obtain 190 mg of Compound (65) in the form of a dark green crystalline product.

¹H-NMR (DMSO-d₆): 9.31 (2H, d), 8.78 (2H, d), 8.00-7.50 (15H, m), 4.54 (2H, d), 1.97 (1H, bs), 1.81-1.40 (5H, m), 1.30-1.00 (5H, m).

### SYNTHESIS EXAMPLE 18

### Preparation of Compound (68)

Compound (68) was prepared in accordance with the following scheme:

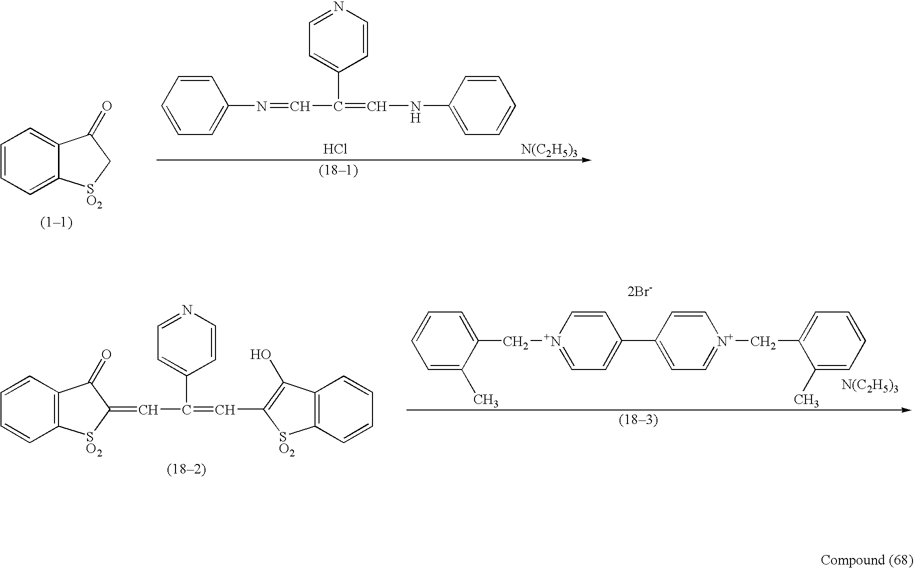

1) Preparation of Compound (18-2)

In 30 mL of N,N-dimethylformamide, 2.00 g of Compound (1-1) and 1.84 g of Compound (18-1) were dispersed. To the liquid stirred at room temperature, 4.59 mL of triethylamine was added. The mixture was stirred for 2 hours at room temperature, and then 150 mL of methanol and 3 mL of concentrated hydrochloric acid were successively added. The resulting mixture was further stirred for 2 hours at room temperature, and the formed precipitate was collected, washed with methanol, and dried to obtain 2.05 g of Compound (18-2) in the form of a dark green crystalline product.

2) Preparation of Compound (67)

In 20 mL of methanol, 200 mg of Compound (18-2) and 110 mg of Compound (18-3) were dispersed. To the liquid stirred at room temperature, 42.4 mg of triethylamine was added. The resulting mixture was stirred for 2 hours at room temperature, and the formed precipitate was collected, washed with methanol, and dried to obtain 205 mg of Compound (68) in the form of a dark green crystalline product.

¹H-NMR (DMSO-d₆): 9.37 (2H, d), 8.77 (2H, d), 8.75 (2H, d), 8.00-7.81 (8H, m), 7.52 (2H, d), 7.49-7.26 (6H, m), 6.02 (2H, s), 2.33 (3H, s).

### SYNTHESIS EXAMPLE 19

### Preparation of Compound (69)

Compound (69) was prepared in accordance with the following scheme:

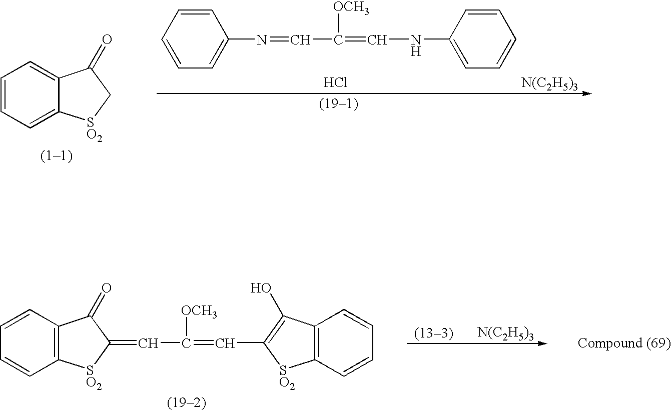

1) Preparation of Compound (19-2)

In 30 mL of N,N-dimethylformamide, 2.00 g of Compound (1-1) and 1.58 g of Compound (19-1) were dispersed. To the liquid stirred at room temperature, 4.59 mL of triethylamine was added. The mixture was stirred for 2.5 hours at room temperature, and then 150 mL of methanol and 3 mL of concentrated hydrochloric acid were successively added. The resulting mixture was further stirred for 2 hours at room temperature, and the formed precipitate was collected, washed with methanol, and dried to obtain 1.95 g of Compound (19-2) in the form of a dark green crystalline product.

2) Preparation of Compound (69)

In 20 mL of methanol, 200 mg of Compound (19-2) and 93.4 mg of Compound (13-3) were dispersed. To the liquid stirred at room temperature, 47.0 mg of triethylamine was added. The resulting mixture was stirred for 2 hours at room temperature, and the formed precipitate was collected, washed with methanol, and dried to obtain 195 mg of Compound (69) in the form of a dark green crystalline product.

¹H-NMR (DMSO-d₆): 9.40 (2H, d), 8.79 (2H, d), 8.02-7.75 (8H, m), 7.55 (2H, s), 4.65 (2H, t), 2.00 (2H, tq), 0.91 (3H, t).

### SYNTHESIS EXAMPLE 20

### Preparation of Compound (71)

Compound (71) was prepared in accordance with the following scheme:

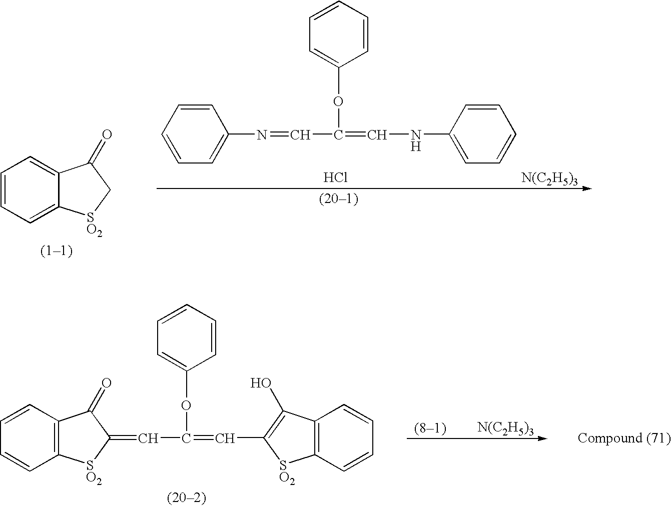

1) Preparation of Compound (20-2)

In 30 mL of N,N-dimethylformamide, 2.00 g of Compound (1-1) and 1.93 g of Compound (20-1) were dispersed. To the liquid stirred at room temperature, 4.59 mL of triethylamine was added. The mixture was stirred for 2.5 hours at room temperature, and then 150 mL of methanol and 3 mL of concentrated hydrochloric acid were successively added. The resulting mixture was further stirred for 2 hours at room temperature, and the formed precipitate was collected, washed with methanol, and dried to obtain 2.15 g of Compound (20-2) in the form of a dark green crystalline product.

2) Preparation of Compound (71)

In 20 mL of methanol, 200 mg of Compound (20-2) and 103 mg of Compound (8-1) were dispersed. To the liquid stirred at room temperature, 41.1 mg of triethylamine was added. The resulting mixture was stirred for 2 hours at room temperature, and the formed precipitate was collected, washed with methanol, and dried to obtain 202 mg of Compound (71) in the form of a dark green crystalline product.

¹H-NMR (DMSO-d₆): 9.47 (2H, d), 8.74 (2H, d), 8.01-7.30 (20H, m), 7.56 (2H, s), 6.51 (2H, s).

The oxonol compound of the formula (1) may be employed singly or in combination with two or more kinds. Further, it may be used in combination with other known dyes conventionally used for information recording medium. Examples of the known dyes include oxonol dyes other than the formula (1), cyanine dyes, phthalocyanine dyes, pyrylium/thiopyrylium dyes, azulenium dyes, squarilium dyes, naphthoquinone dyes, triphenylmethane dyes, and triallylmethane dyes.

The information recording medium of the invention comprises a substrate and a recording layer provided thereon, and the recording layer comprises a dye compound represented by the formula (1). Preferably a light-reflecting layer is provided on the recording layer, and more preferably a protective layer is further provided on the light-reflecting layer.

The information recording medium of the invention can be prepared, for example, in the following manner.

The substrate (support) can be made of any of materials known as those for producing the substrate of the known optical information recording medium. Examples of the materials include glass, polycarbonate, acrylic resins such as polymethyl methacrylate, vinyl chloride resins such as polyvinyl chloride and vinyl chloride copolymer, epoxy resins, amorphous polyolefins, and polyesters. These materials can be employed in combination, if desired. The material is molded to give a film or a rigid plate. Polycarbonate is preferred from the viewpoints of anti-humidity, dimensional stability and cost performance.

The substrate may have an undercoating layer on its surface of the recording layer side, so as to enhance surface smoothness and adhesion and to keep the recording layer from deterioration. Examples of the materials for the undercoating layer include polymers (e.g., polymethyl methacrylate, acrylate-methacrylate copolymer, styrene-maleic anhydride copolymer, polyvinyl alcohol, N-methyl-olacrylamide, styrene-vinyltoluene copolymer, chloro-sulfonated polyethylene, nitrocellulose, polyvinyl chloride, chlorinated polyolefin, polyester, polyimide, vinyl acetate-vinyl chloride copolymer, ethylene-vinyl acetate copolymer, polyethylene, polypropylene, polycarbonate), and surface treating agents such as a silane-coupling agent.

The undercoating layer can be formed by coating a coating solution (in which one or more of the above-mentioned materials are dissolved or dispersed) on the surface of the substrate by the known coating methods such as spin-coat, dip-coat, and extrusion-coat. The undercoating layer generally has a thickness of 0.005 to 20 μm, preferably 0.01 to 10 μm.

On the surface of the substrate or on the undercoating layer, a pregroove for tracking or giving address signals is formed. The pregroove is preferably formed directly on the surface of the substrate when the substrate is molded from polymer material by injection or extrusion.

Alternatively, the pregroove can be provided on the surface of the substrate by placing a pregroove layer. The pregroove layer can be produced from a mixture of a monomer (such as monoester, diester, triester and tetraester) of acrylic acid (or its oligomer) and a photo-polymerization reaction initiator. The pregroove layer can be produced by the steps of coating a precisely produced stamper with the mixture of the polymerization initiator and the monomer such as the above-mentioned acrylic ester, placing a substrate on the coated layer, and irradiating the coated layer with ultra-violet rays through the stamper or the substrate so as to cure the coated layer as well as to combine the cured layer and the substrate. The substrate to which the cured coated layer is attached is separated from the stamper, to give the desired substrate equipped with the pregroove layer. The thickness of the pregroove layer generally is in the range of 0.05 to 100 μm, preferably in the range of 0.1 to 50 μm.

The pregroove formed on the substrate preferably has a depth in the range of 300 to 2000 angstroms and a half-width of 0.2 to 0.9 μm. A depth of 1500 to 2000 angstroms of the pregroove is preferably adopted because such pregroove can enhance the sensitivity without decreasing the light-reflection on the substrate. The optical disc (in which a recording layer containing the oxonol dye of the formula (1) and a light-reflection layer are provided on the deep pregroove in order) shows a high sensitivity, and hence is employable even in a recording system using a laser beam of a low power. This means that a semiconductor laser of a low output power can be employed, and the life of semiconductor laser can be prolonged.

On the substrate (or the undercoating layer) provided with the pregroove, the recording layer comprising the dye compound of the formula (1) is placed.

The recording layer may contain a compound known as a singlet oxygen quencher. Examples of the quencher include the metal complexes, the diimmonium salts, and the aminium salts of the formulas (III), (IV) and (V), respectively, in Japanese Patent Provisional Publication No. 3(1991)-224793, and nitroso compounds described in Japanese Patent Provisional Publications No. 2(1990)-300287 and No. 2(1990)-300288.

The recording layer can be formed on the substrate (support) by the steps of dissolving the oxonol dye of the formula (1) and, if desired, the quencher and a binder in a solvent to prepare a coating liquid, applying the liquid onto the substrate (or the undercoating layer) to form a layer, and then drying the formed layer.

Examples of the solvents for the coating liquid include esters (e.g., butyl acetate and cellosolve acetate), ketones (e.g., methyl ethyl ketone, cyclohexanone, and methyl isobutyl ketone), chlorinated hydrocarbons (e.g., dichloromethane, 1,2-dichloroethane, and chloroform), amides (e.g., dimethylformamide), hydrocarbons (e.g., cyclohexanone), ethers (e.g., tetrahydrofuran, diethyl ether, and dioxane), alcohols (e.g., ethanol, n-propanol, isopropanol, n-butanol, and diacetone alcohol), fluorine-containing solvents (e.g., 2,2,3,3-tetrafluoropropanol), and glycol ethers (e.g., ethyleneglycol monomethyl ether, ethyleneglycol monoethyl ether, and pro-pyleneglycol monomethyl ether). These solvents may be employed singly or in combination, in consideration of the solubility of the used compound in the solvent. The coating liquid can further contain auxiliary additives such as an oxidation inhibitor, a UV absorber, a plasticizer, and a lubricant.

Examples of the binders include natural-origin polymers (e.g., gelatin, cellulose derivatives, dextran, rosin, and rubber), hydrocarbon polymer resins (e.g., polyethylene, polypropylene, polystyrene, and polyisobutyrene), vinyl polymers (e.g., polyvinyl chloride, polyvinylidene chloride, and vinyl chloride-vinyl acetate copolymer), acrylate polymers (e.g., polymethyl acrylate and polymethyl methacrylate), polyvinyl alcohol, chlorinated polyethylene, epoxy resins, butyral resins, rubber derivatives, and thermosetting resins (e.g., prepolymers of phenol-formaldehyde). The binder for forming the recording layer is employed so that a weight ratio of the binder to the dye may be in the range of not more than 10/1, preferably not more than 1/1, more preferably not more than 1/10. The concentration of the coating liquid thus prepared is generally in the range of 0.01 to 10 wt. %, preferably 0.1 to 5 wt. %.

The coating procedure can be performed by the known methods such as spray coat, spin coat, dip coat, roller coat, blade coat, doctor roller coat, and screen print. The recording layer may be a single layer or may comprise plural layers, and the thickness is generally in the range of 20 to 500 nm (preferably 50 to 300 nm). The recording layer may be provided on both of the surfaces of the substrate.

On the recording layer, a light-reflecting layer is usually placed so as to enhance the light-reflection in the course of reproduction of information.

The light-reflecting material for the light-reflecting layer should show a high reflection to the laser light. Examples of the light-reflecting materials include metals and sub-metals such as Mg, Se, Y, Ti, Zr, Hf, V, Nb, Ta, Cr, Mo, W, Mn, Re, Fe, Co, Ni, Ru, Rh, Pd, Ir, Pt, Cu, Ag, Au, Zn, Cd, Al, Ga, In, Si, Ge, Te, Pb, Po, Sn, and Bi. Stainless steel film is also employable. Particularly preferred are Cr, Ni, Pt, Cu, Ag, Au, Al and stainless steel. These materials can be employed singly, in combination, or in the form of alloy.

The light-reflecting layer can be formed on the recording layer by, for example, vacuum deposition, sputtering or ion-plating. The thickness of the light-reflecting layer is generally 10 to 300 nm, preferably 50 to 200 nm.

On the light-reflecting layer, a protective layer is preferably provided so as to protect the recording layer and the light-reflecting layer from chemical deterioration or physical shock. Besides on the light-reflecting layer, another protective layer can be also placed on the substrate on the face not having the dye recording layer so as to enhance the scratch resistance and the moisture resistance of the medium. The protective layer can be made of inorganic materials (e.g., SiO, SiO₂, MgF₂, SnO₂, Si₃N₄) or organic materials (e.g., thermo-plastic resins, thermo-setting resins, UV curable resins).

On the light-reflecting layer and/or the substrate, the protective layer can be formed by laminating a film of plastic material using an adhesive. The inorganic material can be also placed on the light-reflecting layer and/or the substrate by vacuum deposition or sputtering. Otherwise, the organic polymer material layer can be formed by the steps of dissolving the polymer material to prepare a coating solution, applying the coating solution to form a layer, and then drying the formed layer to give the protective layer. For example, a UV curable resin is dissolved in a solvent and applied on the light-reflecting layer and/or the substrate, and then cured by applying ultraviolet rays to the formed layer. The protective layer can contain various additives such as an anti-static agent and an oxidation inhibitor. The thickness of the protective layer generally is in the range of 0.1 to 100 μm.

The information recording medium of the invention may comprise one substrate. However, the invention can be applied for a laminated type medium. The laminated type medium can be prepared by combining two substrates (at least one of which has the structure of the invention) with adhesive so that the recording dye layer may be positioned inside. If the two substrates are laminated via spacer rings (an inner spacer ring and an outer spacer ring), an air-sandwich type medium can be obtained.

Since the recording medium of the invention shows high light-reflection (approx. 70%), the reproduction of the recorded information can be accomplished by means of a commercially available CD player.

The information is, for example, recorded on the medium of the invention in the following manner.

The information recording medium is made to rotate at a constant line rate [1.2 to 14 m/sec., in the case of CD format] or a constant angle rate, or at a twofold or more rate. On the rotating medium, a recording light source such as a semiconductor laser beam is applied through the transparent substrate. By the application of the laser beam, pits are formed in the recording dye layer. The pit is assumed to be produced of local vacancy of the recording layer, deformation of the recording layer and/or the substrate, and change of the physical or chemical characteristics of the recording layer. The light source preferably is a semiconductor laser having an oscillation frequency of 500 to 850 nm. The preferred beam wavelength is in the range of 500 to 800 nm. In the case of the CR-R type recording medium, the laser beam preferably has a wavelength of 770 to 790 nm.

The reproduction of the recorded information can be accomplished by applying a semiconductor laser beam through the substrate on the medium rotating at the above line rate or at the twofold or more rate. The light-reflection is then detected from the substrate side.

The present invention is further described by the following non-restrictive working examples.

### EXAMPLE 1

In 100 mL of 2,2,3,3-tetrafluoropropanol, 3 g of Compound (1) was dissolved to prepare a coating solution for recording layer. The coating liquid was then applied by spin-coat on the surface (on which pregroove was beforehand formed) of a transparent polycarbonate substrate (diameter: 120 mm, thickness: 1.2 mm) to give a recording layer (average thickness at the pregroove: approx. 200 nm). The pregroove had been beforehand formed spirally on the substrate (track pitch: 1.7 μm, pregroove width: 0.4 μm, depth: 0.16 μm) by injection molding of the substrate.

On the coated dye recording layer, a light-reflecting layer of Ag (thickness: about 100 nm) was provided by sputtering. Subsequently, a UV curable photopolymer (SD-220 [trade name], available from Dainippon Ink & Chemicals, Inc.) was applied on the light-reflecting layer, and then irradiated with ultra-violet rays for curing to form a protective layer of 5 μm thick. Thus, an information recording medium (CD-R) of the invention was produced.

### EXAMPLES 2-24

The procedure of Example 1 was repeated except that each oxonol compound shown in Table 1 or 2 was used in place of Compound (1), to produce an information recording medium (CD-R) of the invention.

### COMPARISON EXAMPLES 1-4

The procedure of Example 1 was repeated except that each of the following dye compounds [comparison dye compounds (A), (B), (C), and (D)] was used in place of Compound (1), to produce an information recording medium (CD-R) for comparison [comparison dye compounds (A), (B), (C), and (D) correspond to Comparison Examples 1 to 4, respectively].

Comparison Dye Compound (A)

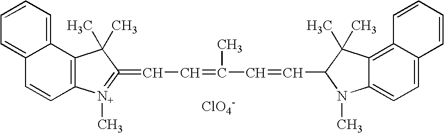

Comparison Dye Compound (B):

Example 24 of Japanese Patent Provisional Publication No. 63(1988)-209995

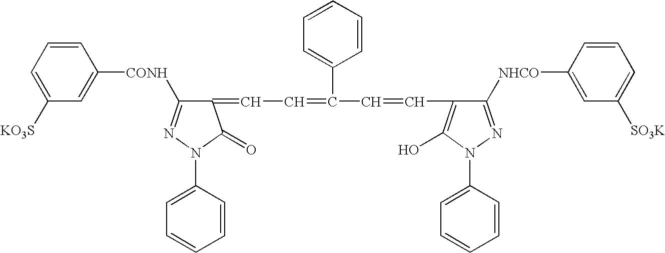

Comparison Dye Compound (C):

Example 28 of Japanese Patent Provisional Publication No. 63(1988)-209995

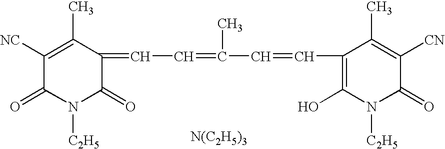

Comparison Dye Compound (D)

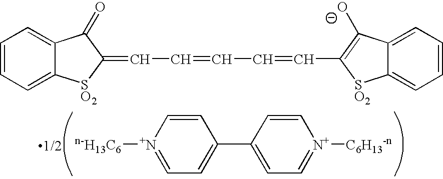

### Evaluation of CD-R Information Recording Medium

(1) Modulation

On each sample disc rotating at 1.4 m/second, EFM signals of 3T and l1T were recorded by means of a semi-conductor laser beam (wavelength: 780 nm). The laser power was varied between 4 mW and 9 mW. Thus recorded signals were then reproduced using a laser beam (laser power: 0.5 mW) to determine the modulations at 3T and 11T under the optimum power.

(2) Light Reflectance

In the above-mentioned reproducing procedure, the light reflectance at the non-recorded pregroove area (on which the signals were not recorded) was determined under the optimum power.

(3) Resistance to Light

Each sample on which the signals had been beforehand recorded in the above-mentioned manner was irradiated with light of a xenon lamp (140,000 Lux) for 18 or 36 hours. After the irradiation was complete, the modulations were measured in the above manner. Further, the color of the irradiated sample disc was observed and classified into the following grades:

AA: not faded,

BB: slightly faded, and

CC: completely faded.

The results are set forth in Tables 1 and 2.

TABLE 1

| | | | |
|---|---|---|---|
| irradiation | 3T modulation | 11T modulation | color fading |

Example 1: Compound (1)

| | | | |
|---|---|---|---|
| before | 0.563 | 0.820 | — |
| after 18 hours | 0.559 | 0.815 | AA |
| after 36 hours | 0.552 | 0.808 | AA |

Example 2: Compound (4)

| | | | |
|---|---|---|---|
| before | 0.554 | 0.803 | — |
| after 18 hours | 0.550 | 0.796 | AA |
| after 36 hours | 0.543 | 0.789 | AA |

Example 3: Compound (7)

| | | | |
|---|---|---|---|
| before | 0.564 | 0.825 | — |
| after 18 hours | 0.560 | 0.817 | AA |
| after 36 hours | 0.556 | 0.809 | AA |

Example 4: Compound (8)

| | | | |
|---|---|---|---|
| before | 0.569 | 0.831 | — |
| after 18 hours | 0.560 | 0.825 | AA |
| after 36 hours | 0.552 | 0.819 | AA |

Example 5: Compound (11)

| | | | |
|---|---|---|---|
| before | 0.559 | 0.822 | — |
| after 18 hours | 0.550 | 0.811 | AA |
| after 36 hours | 0.545 | 0.799 | AA |

Example 6: Compound (16)

| | | | |
|---|---|---|---|
| before | 0.531 | 0.821 | — |
| after 18 hours | 0.527 | 0.815 | AA |
| after 36 hours | 0.521 | 0.808 | AA |

Example 7: Compound (17)

| | | | |
|---|---|---|---|
| before | 0.535 | 0.825 | — |
| after 18 hours | 0.529 | 0.821 | AA |
| after 36 hours | 0.519 | 0.811 | AA |

Example 8: Compound (20)

| | | | |
|---|---|---|---|
| before | 0.529 | 0.819 | — |
| after 18 hours | 0.521 | 0.812 | AA |
| after 36 hours | 0.516 | 0.802 | AA |

Example 9: Compound (22)

| | | | |
|---|---|---|---|
| before | 0.501 | 0.802 | — |
| after 18 hours | 0.495 | 0.797 | AA |
| after 36 hours | 0.488 | 0.789 | AA |

Example 10: Compound (26)

| | | | |
|---|---|---|---|
| before | 0.562 | 0.813 | — |
| after 18 hours | 0.213 | 0.302 | BB |

| | | |
|---|---|---|
| after 36 hours | tracking error | CC |

Example 11: Compound (28)

| | | | |
|---|---|---|---|
| before | 0.556 | 0.802 | — |
| after 18 hours | 0.551 | 0.798 | BB |

| | | |
|---|---|---|
| after 36 hours | tracking error | CC |

Example 12: Compound (31)

| | | | |
|---|---|---|---|
| before | 0.560 | 0.810 | — |
| after 18 hours | 0.551 | 0.802 | AA |
| after 36 hours | 0.535 | 0.793 | AA |

Example 13: Compound (32)

| | | | |
|---|---|---|---|
| before | 0.545 | 0.815 | — |
| after 18 hours | 0.538 | 0.811 | AA |
| after 36 hours | 0.529 | 0.799 | AA |

Example 14: Compound (35)

| | | | |
|---|---|---|---|
| before | 0.528 | 0.800 | — |
| after 18 hours | 0.521 | 0.798 | AA |
| after 36 hours | 0.513 | 0.790 | AA |

Example 15: Compound (36)

| | | | |
|---|---|---|---|
| before | 0.560 | 0.812 | — |
| after 18 hours | 0.555 | 0.803 | AA |
| after 36 hours | 0.550 | 0.797 | AA |

Example 16: Compound (37)

| | | | |
|---|---|---|---|
| before | 0.555 | 0.815 | — |
| after 18 hours | 0.550 | 0.809 | AA |
| after 36 hours | 0.541 | 0.800 | AA |

Example 17: Compound (41)

| | | | |
|---|---|---|---|
| before | 0.548 | 0.812 | — |
| after 18 hours | 0.225 | 0.315 | BB |

| | | |
|---|---|---|
| after 36 hours | tracking error | CC |

Example 18: Compound (42)

| | | | |
|---|---|---|---|
| before | 0.552 | 0.811 | — |
| after 18 hours | 0.540 | 0.805 | AA |
| after 36 hours | 0.533 | 0.798 | AA |

Example 19: Compound (44)

| | | | |
|---|---|---|---|
| before | 0.555 | 0.805 | — |
| after 18 hours | 0.551 | 0.801 | AA |
| after 36 hours | 0.536 | 0.793 | AA |

TABLE 2

| | | | |
|---|---|---|---|
| irradiation | 3T modulation | 11T modulation | color fading |

Example 20: Compound (46)

| | | | |
|---|---|---|---|
| before | 0.541 | 0.800 | — |
| after 18 hours | 0.538 | 0.795 | AA |
| after 36 hours | 0.531 | 0.790 | AA |

Example 21: Compound (49)

| | | | |
|---|---|---|---|
| before | 0.540 | 0.801 | — |
| after 18 hours | 0.535 | 0.796 | AA |
| after 36 hours | 0.530 | 0.790 | AA |

Example 22: Compound (51)

| | | | |
|---|---|---|---|
| before | 0.540 | 0.801 | — |
| after 18 hours | 0.535 | 0.800 | AA |
| after 36 hours | 0.529 | 0.795 | AA |

Example 23: Compound (56)

| | | | |
|---|---|---|---|
| before | 0.495 | 0.798 | — |
| after 18 hours | 0.490 | 0.791 | AA |
| after 36 hours | 0.482 | 0.779 | AA |

Example 24: Compound (58)

| | | | |
|---|---|---|---|
| before | 0.494 | 0.800 | — |
| after 18 hours | 0.395 | 0.659 | BB |

| | | |
|---|---|---|
| after 36 hours | tracking error | CC |

Comparison Example 1: Comparison dye compound (A)

| | | | |
|---|---|---|---|
| before | 0.560 | 0.811 | — |

| | | |
|---|---|---|
| after 18 hours | tracking error | CC |
| after 36 hours | tracking error | CC |

Comparison Example 2: Comparison dye compound (B)

| | | | |
|---|---|---|---|
| before | 0.233 | 0.380 | — |
| after 18 hours | 0.101 | 0.292 | CC |

| | | |
|---|---|---|
| after 36 hours | tracking error | CC |

Comparison Example 3: Comparison dye compound (C)

| | | | |
|---|---|---|---|
| before | 0.256 | 0.387 | — |
| after 18 hours | 0.103 | 0.295 | CC |

| | | |
|---|---|---|
| after 36 hours | tracking error | CC |

Comparison Example 4: Comparison dye compound (D)

| | | | |
|---|---|---|---|
| before | 0.293 | 0.402 | — |
| after 18 hours | 0.118 | 0.306 | BB |

| | | |
|---|---|---|
| after 36 hours | tracking error | CC |

The results shown in Tables 1 and 2 indicate that each CD-R media of the invention have both excellent recording/reproducing characteristics and strong light resistance, as compared with the CD-R media of Comparison examples.

### EXAMPLE 25

In 100 mL of 2,2,3,3-tetrafluoropropanol, 3 g of Compound (61) was dissolved to prepare a coating solution for recording layer. The coating liquid was then applied by spin-coat on the surface (on which pregroove was formed) of a transparent polycarbonate substrate (diameter: 120 mm, thickness: 0.6 mm) to give a recording layer (average thickness at the pregroove: approx. 200 nm). The pregroove was beforehand formed spirally on the substrate (track pitch: 0.8 μm, pregroove width: 0.4 μm, depth: 0.15 μm) by injection molding of the substrate.

On the coated dye recording layer, a light-reflecting layer of Ag (thickness: about 100 nm) was provided by sputtering.

The formed disc and another polycarbonate substrate (a protective disc substrate having the diameter of 120 mm and the thickness of 0.6 mm) are laminated with adhesive so that the recording layer may be positioned inside. Thus, an information recording medium of DVD-R type (thickness: approx. 1.2 mm) of the invention was produced.

### EXAMPLES 26-31

The procedure of Example 25 was repeated except that each oxonol compound shown in Table 3 was used in place of Compound (61), to produce an information recording medium (DVD-R) of the invention.

### COMPARISON EXAMPLES 5-8

The procedure of Example 25 was repeated except that each of the following dye compounds [comparison dye compounds (E), (F), (G), and (H)] was used in place of Compound (61), to produce an information recording medium (DVD-R) for comparison [comparison dye compounds (E), (F), (G), and (H) correspond to Comparison Examples 5 to 8, respectively].

Comparison Dye Compound (E)

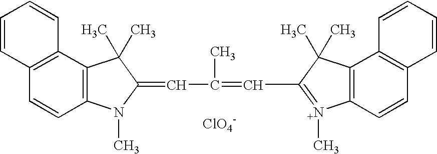

Comparison Dye Compound (F)

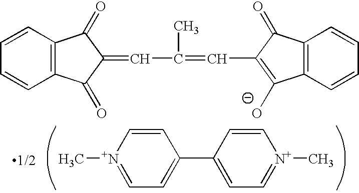

Comparison Dye Compound (G)

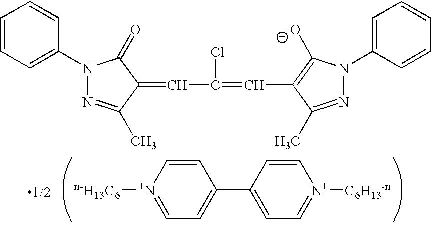

Comparison Dye Compound (H)

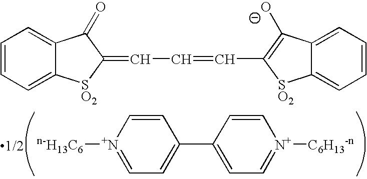

### Evaluation of DVD-R Information Recording Medium

(1) Modulation

On each sample rotating at 3.8 m/second, EFM signals of 3T and 14T were recorded by means of a semiconductor laser beam (wavelength: 635 nm). The laser power was varied between 4 mW and 9 mW. Thus recorded signals were then reproduced using a laser beam (laser power: 0.5 mW) to determine the modulations at 3T and 14T under the optimum power.

(2) Light Reflectance

In the above reproducing procedure, the light reflectance at the non-recorded pregroove area (on which the signals were not recorded) was determined under the optimum power.

(3) Resistance to Light

Each sample on which the signals had been beforehand recorded in the above manner was irradiated with light of a xenon lamp (140,000 Lux) for 18 or 36 hours. After the irradiation was complete, the modulations were determined in the above-mentioned manner. Further, the color of the sample disc was observed and classified into the following grades:

AA: not faded,

BB: slightly faded, and

CC: completely faded.

The results are set forth in Table 3.

TABLE 3

| | | | |
|---|---|---|---|
| irradiation | 3T modulation | 14T modulation | color fading |

Example 25: Compound (61)

| | | | |
|---|---|---|---|
| before | 0.333 | 0.811 | — |
| after 18 hours | 0.325 | 0.802 | AA |
| after 36 hours | 0.321 | 0.798 | AA |

Example 26: Compound (63)

| | | | |
|---|---|---|---|
| before | 0.312 | 0.795 | — |
| after 18 hours | 0.309 | 0.785 | AA |
| after 36 hours | 0.297 | 0.776 | AA |

Example 27: Compound (65)

| | | | |
|---|---|---|---|
| before | 0.352 | 0.820 | — |
| after 18 hours | 0.344 | 0.813 | AA |
| after 36 hours | 0.340 | 0.805 | AA |

Example 28: Compound (68)

| | | | |
|---|---|---|---|
| before | 0.304 | 0.778 | — |
| after 18 hours | 0.298 | 0.772 | AA |
| after 36 hours | 0.288 | 0.765 | AA |

Example 29: Compound (69)

| | | | |
|---|---|---|---|
| before | 0.299 | 0.765 | — |
| after 18 hours | 0.284 | 0.760 | AA |
| after 36 hours | 0.279 | 0.752 | AA |

Example 30: Compound (71)

| | | | |
|---|---|---|---|
| before | 0.307 | 0.764 | — |
| after 18 hours | 0.295 | 0.761 | AA |
| after 36 hours | 0.286 | 0.754 | AA |

Example 31: Compound (73)

| | | | |
|---|---|---|---|
| before | 0.339 | 0.805 | — |
| after 18 hours | 0.331 | 0.799 | AA |
| after 36 hours | 0.326 | 0.797 | AA |

Comparison Example 5: Comparison dye compound (E)

| | | | |
|---|---|---|---|
| before | 0.343 | 0.801 | — |
| after 18 hours | tracking error | | CC |
| after 36 hours | tracking error | | CC |

Comparison Example 6: Comparison dye compound (F)

| | | | |
|---|---|---|---|
| before | 0.204 | 0.480 | — |
| after 18 hours | 0.189 | 0.449 | BB |
| after 36 hours | 0.182 | 0.388 | BB |

Comparison Example 7: Comparison dye compound (G)

| | | | |
|---|---|---|---|
| before | 0.191 | 0.476 | — |
| after 18 hours | 0.183 | 0.442 | BB |
| after 36 hours | 0.177 | 0.379 | BB |

Comparison Example 8: Comparison dye compound (H)

| | | | |
|---|---|---|---|
| before | 0.253 | 0.498 | — |
| after 18 hours | 0.246 | 0.483 | AA |
| after 36 hours | 0.241 | 0.475 | AA |

The results shown in Table 3 also indicate that each DVD-R medium of the invention has both excellent recording/reproducing characteristics and strong light resistance, as compared with the DVD-R media of Comparison examples.

What is claimed is:

1. An information recording medium comprising a substrate and a recording layer provided thereon on which information can be recorded by irradiation with a laser beam, wherein the recording layer comprises an oxonol compound of the following formula (1) in which at least one carbon atom of the methine chain comprises a substituent group of (R³)ᵣ: 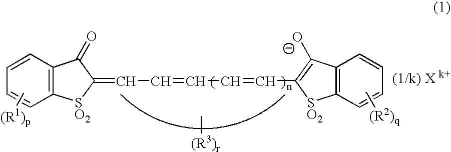
in which each of R¹, R² and R³ independently represents a substituent group, X^{k+} represents an onium ion, each of p and q independently is an integer of 0 to 4, r is an integer of 1 to 5, n is 0 or 1, k is an integer of 1 to 10, and in the case where p or q is an integer of 2 or more, plural substituent groups represented by R¹ or R² are the same or different or they can connect with each other to form a ring.

2. The information recording medium of claim 1, wherein each of R¹, R² and R³ of the formula (1) independently is an alkyl group having 1-18 carbon atoms, an alkenyl group having 2-18 carbon atoms, an alkynyl group having 2-18 carbon atoms, an aryl group having 6-14 carbon atoms, a heterocyclic group having 4-9 carbon atoms, a halogen atom, nitro group, cyano group, or a group represented by —COR¹¹, —SO₂R¹¹, —SOR¹¹, —CO₂R¹¹, —OR¹¹, —SR¹¹, —OCOR¹¹, —OSO₂R¹¹, —CONR¹¹R¹², —SO₂NR¹¹R¹², —OCONR¹¹R¹², —OSO₂N—R¹¹R¹², —NR¹¹R¹², —NR¹¹COR¹², —NR¹¹SO₂R¹², —NR¹¹CO₂R¹², —NR¹¹CONR¹²—R¹³, or —NR¹¹SO₂NR¹²R¹³ in which each of R¹¹, R¹² and R¹³ independently represents an alkyl group having 1-18 carbon atoms, an alkenyl group having 2-18 carbon atoms, an alkynyl group having 2-18 carbon atoms, an aryl group having 6-14 carbon atoms, or a heterocyclic group having 4-9 carbon atoms.

3. The information recording medium of claim 1, wherein X^{k+} is a quaternary ammonium ion.

4. The information recording medium of claim 1, wherein X^{k+} is an onium ion of the following formula (2): 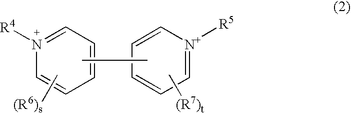
in which each of R⁴ and R⁵ independently represents an alkyl group, an alkenyl group, an alkynyl group, an aryl group, or a heterocyclic group; each of R⁶ and R⁷ independently represents a substituent group; each of s and t independently represents an integer of 0 to 4; and in the case where s or t is an integer of 2 or more, plural substituent groups represented by R⁶ or R⁷ is the same or different or they can connect with each other to form a ring.

5. The information recording medium of claim 1, wherein a light-reflecting layer is further provided on the recording layer.

6. An oxonol compound having the following formula (3) 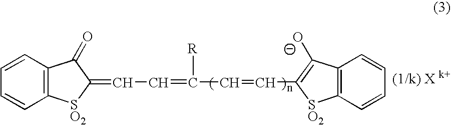
in which R represents a substituent group selected from the group consisting of methyl, ethyl, phenyl, 4-pyridyl, methoxy, and phenoxy; n is 0 or 1, X^{k+} represents an onium ion; and k is 1 or 2.

7. An information recording medium comprising a substrate and a recording layer provided thereon on which information can be recorded by irradiation with a laser beam, wherein the recording layer comprises an oxonol compound of claim 6.
